(19) United States
(12) Patent Application Publication
(10) Pub. No.: US 2025/0160862 A1
(43) Pub. Date: May 22, 2025

(54) Low-profile shock wave catheters

(71) Applicant: Shockwave Medical, Inc., Santa Clara, CA (US)

(72) Inventors: Rainier BETELIA, San Jose, CA (US); Danielle Sheree ROND, Santa Clara, CA (US); Todd Weston JENKINS, San Jose, CA (US); Nancy P. NGUYEN, San Jose, CA (US)

(73) Assignee: Shockwave Medical, Inc., Santa Clara, CA (US)

(21) Appl. No.: 18/949,806

(22) Filed: Nov. 15, 2024

Related U.S. Application Data
(60) Provisional application No. 63/599,950, filed on Nov. 16, 2023.

(51) Int. Cl. A61B 17/22; A61B 17/00; A61M 25/00

(52) U.S. Cl. CPC A61B 17/22012; A61M 25/001; A61B 2017/00526; A61B 2017/22025

(57) ABSTRACT

Described herein are shock wave catheters having discontinuous emitter bands. The catheters may be used for treating occlusions in a body lumen. In some examples, the catheters include an elongated tube, a shock wave electrode assembly at least partially embedded within the elongated tube, and an enclosure surrounding the shock wave electrode assembly. In some examples, the electrode assembly includes an emitter band at least partially encircling the elongated tube, the emitter band forming a first electrode of an electrode pair, and one wire having a distal end forming a second electrode of the electrode pair. The shock wave electrode assembly is configured to emit a shock wave when a voltage pulse is applied across the electrode pair. In some examples, the at least one emitter band is compressible to reduce a diameter of the emitter band.

### CROSS REFERENCE TO RELATED APPLICATIONS

This application claims priority benefit of U.S. Provisional Application No. 63/599,950, filed on Nov. 16, 2023, the entire contents of which are incorporated herein by reference.

### FIELD OF THE DISCLOSURE

The present disclosure relates generally to the field of medical devices and methods, and more specifically to shock wave catheter devices for treating calcified lesions in body lumens, such as calcified lesions and occlusions in vasculature and kidney stones in the urinary system.

### BACKGROUND

A wide variety of catheters have been developed for treating calcified lesions, such as calcified lesions in vasculature associated with arterial disease. For example, treatment systems for percutaneous coronary angioplasty or peripheral angioplasty use angioplasty balloons to dilate a calcified lesion and restore normal blood flow in a vessel. In these types of procedures, a catheter carrying a balloon is advanced into the vasculature along a guide wire until the balloon is aligned with calcified plaques. The balloon is then pressurized (normally to greater than 10 atm), causing the balloon to expand in a vessel to push calcified plaques back into the vessel wall and dilate occluded regions of vasculature.

More recently, the technique and treatment of intravascular lithotripsy (IVL) has been developed, which is an interventional procedure to modify calcified plaque in diseased arteries. The mechanism of plaque modification is through use of a catheter having one or more acoustic shock wave generating sources that can generate acoustic shock waves that modify the calcified plaque. IVL devices vary in design with respect to the energy source used to generate the acoustic shock waves, with two exemplary energy sources being electrohydraulic generation and laser generation.

For electrohydraulic generation of acoustic shock waves, a conductive solution (e.g., saline) may be contained within an enclosure that surrounds electrodes or can be flushed through a tube that surrounds the electrodes. The calcified plaque modification is achieved by creating acoustic shock waves within the catheter by an electrical discharge across the electrodes. This discharge creates one or more rapidly expanding vapor bubbles that generate the acoustic shock waves. These shock waves propagate radially outward and modify calcified plaque within the blood vessels. For laser generation of acoustic shock waves, a laser pulse is transmitted into and absorbed by a fluid within the catheter. This absorption process rapidly heats and vaporizes the fluid, thereby generating the rapidly expanding vapor bubble, as well as the acoustic shock waves that propagate outward and modify the calcified plaque. The acoustic shock wave intensity is higher if a fluid is chosen that exhibits strong absorption at the laser wavelength that is employed. These examples of IVL devices are not intended to be a comprehensive list of potential energy sources to create IVL shock waves.

The IVL process may be considered different from standard atherectomy procedures in that it cracks calcium but does not liberate the cracked calcium from the tissue. Hence, generally speaking, IVL should not require aspiration nor embolic protection. Further, due to the compliance of a normal blood vessel and non-calcified plaque, the shock waves produced by IVL do not modify the normal vessel or non-calcified plaque.

More specifically, catheters to deliver IVL therapy have been developed that include pairs of electrodes for electrohydraulically generating shock waves inside an angioplasty balloon. Shock wave devices can be particularly effective for treating calcified plaque lesions because the acoustic pressure from the shock waves can crack and disrupt lesions near the angioplasty balloon without harming the surrounding tissue. In these devices, the catheter is advanced over a guide wire through a patient's vasculature until it is positioned proximal to and/or aligned with a calcified plaque lesion in a body lumen. The balloon is then inflated with conductive fluid (using a relatively low pressure of 2-4 atm) so that the balloon expands to contact the lesion, but is not an inflation pressure that substantively displaces the lesion. Voltage pulses can then be applied across the electrodes of the electrode pairs to produce acoustic shock waves that propagate through the walls of the angioplasty balloon and into the lesions. Once the lesions have been cracked by the acoustic shock waves, the balloon can be expanded further to increase the cross-sectional area of the lumen and improve blood flow through the lumen. Alternative devices to deliver IVL therapy can be within a closed volume other than an angioplasty balloon, such as a cap, balloons of variable compliancy, or other enclosure.

Efforts have been made to improve the delivery of shock waves in these devices. For instance, forward-biased designs, such as the designs found in U.S. Pat. No. 10,966,737 and U.S. Publication No. 2019/0388110, both of which are incorporated herein by reference, direct shock waves in a generally forward direction (e.g., distally from the distal end of a catheter) to break up tighter and harder-to-cross occlusions in vasculature. Other catheter devices have been designed to include arrays of low-profile electrode assemblies that reduce the crossing profile of the catheter and allow the catheter to more easily navigate calcified vessels to deliver shock waves in more severely occluded regions of vasculature. For instance, U.S. Pat. Nos. 8,888,788, and 10,709,462 and U.S. Publication No. 2021/0085347, each of which is incorporated herein by reference, provide examples of low-profile electrode assemblies. Such forward-biased and low-profile designs are particularly useful when an artery is totally or partially occluded, for example, with thrombus, plaque, fibrous plaque, and/or calcium deposits. When treating such conditions, a physician must first cross the occlusion (e.g., pass through the occluded area), and then feed the angioplasty balloon and/or other tools down the artery to the blockage to perform the desired procedure. In some instances, however, such as the case of a chronic total occlusion (“CTO”), the occlusion may be so tight and solid that it is difficult to cross the treatment device into the true lumen of the distal vessel. Some physicians may implement atherectomy procedures (e.g., laser-based, mechanically cutting or shaving, and/or mechanically rotating devices) to form a channel in a CTO in combination with an angioplasty balloon treatment, but many atherectomy devices and systems carry a higher risk of vessel perforation or vessel dissection as compared with a basic angioplasty balloon catheters.

Despite these advances, conventional shock wave catheters can encounter challenges navigating tight regions of vasculature. The navigability of catheters in tight vessels and advanced or irregular occlusions is limited by the crossing profile of the distal end of the catheter. While conventional lower-profile designs may have crossing profiles that improve navigability, such designs may still have insufficiently low crossing profiles to navigate tighter and/or more severely occluded vessels. Further, the maneuverability of conventional catheters through tight vessels may be restricted by the limited flexibility of components used to generate shock waves.

### SUMMARY

According to some aspects of the present disclosure, a shock wave catheter includes at least one discontinuous emitter band located at or along the distal end of the catheter body. The discontinuous shape of the emitter band allows for the emitter band to fit catheters of a wide range of diameters. In contrast to a ring or other continuous band structure, a discontinuous band structure can provide for greater flexibility of the distal portion of the catheter body because the width of the band does not constitute a length along the catheter which is rigid and inflexible. Rather, where each discontinuous band structure at least partially surrounds the catheter lumen, a section of the catheter lumen will remain uncovered and thus flexible, allowing for a degree of curvature at the location of the discontinuous band structure. Such flexibility and curvature may be less than the possible flexibility and curvature of an unrestrained lumen, but will be greater than the possible flexibility and curvature of a lumen with a solid and complete emitter band ring surrounding a section of lumen. Accordingly, a catheter having one or more discontinuous emitter bands can achieve greater flexibility than would be possible with continuous or annular emitter bands.

Moreover, various designs of discontinuous emitter bands allow the catheter to achieve a smaller crossing profile than would be achievable with a continuous emitter band. A catheter can be assembled in a layered process that includes positioning at least one discontinuous emitter band around a first tube that will form the inner layer of the catheter body of the assembled catheter and then compressing the at least one discontinuous emitter band from an initial uncompressed diameter to a final compressed diameter. Further, a baseline size of discontinuous emitter band may be useable for catheters of different diameters, where the discontinuous emitter band can be compressed or crimped to form around a catheter and surround from 90 degrees to greater than 360 degrees of the catheter circumference, depending on the individual size of the catheter. The at least one discontinuous emitter band may further be embedded into the catheter body to immobilize the emitter band. The smaller crossing profile and improved navigability achieved by such catheters may facilitate the treatment of smaller body lumens, such as tight blood vessels and valves.

According to an aspect, a catheter for treating an occlusion in a body lumen includes an elongated tube; a shock wave electrode assembly at least partially embedded within the elongated tube, the electrode assembly comprising: at least one discontinuous emitter band, the at least one emitter band forming a first electrode of an electrode pair, and at least one wire, a distal end of the at least one wire forming a second electrode of the electrode pair, wherein the shock wave electrode assembly is configured to emit a shock wave when a voltage pulse is applied across the electrode pair; and an enclosure surrounding the shock wave electrode assembly. Optionally, the discontinuity of the at least one discontinuous emitter band enables the at least one emitter band to have circumferential flexibility.

The at least one discontinuous emitter band may include a first end and a second end separated by a gap, and wherein a connecting region between the first end and the second end at least partially encircles an inner layer of the elongated tube. The at least one discontinuous emitter band may encircle at least 180 degrees of the inner layer.

The at least one discontinuous emitter band may include an outer edge, wherein the outer edge of the at least one discontinuous emitter band forms the first electrode of the electrode pair. The distal end of the at least one wire may be positioned proximate to the outer edge of the at least one discontinuous emitter band.

The at least one discontinuous emitter band may include a first enlarged region, a second enlarged region, and at least one connecting region extending therebetween. A width of at least one of the first enlarged region and the second enlarged region may be greater than a width of the at least one connecting region. The at least one discontinuous emitter band may include an aperture extending through the first enlarged region. The aperture may have an elliptical shape. An edge of the aperture may form the first electrode of the electrode pair. The distal end of the at least one wire may be positioned proximate to the edge of the aperture. The at least one discontinuous emitter band may form a spiral coil shape. At least one of the first enlarged region or the second enlarged region may include one or more slots for attaching one or more wires to the at least one discontinuous emitter band.

The elongated tube may include an inner layer and an outer layer, the outer layer at least partially surrounding the at least one discontinuous emitter band. A hole in the outer layer may provide a spark gap between the first electrode and the second electrode of the electrode pair. The outer layer may overlay at least a portion of an outer surface of the at least one discontinuous emitter band. The outer layer may encapsulate at least a portion of the at least one wire. An outer diameter of the outer layer may be equal to an outer diameter of the at least one discontinuous emitter band. The inner layer may be formed of a first polymer, the outer layer may be formed of a second polymer, and the first polymer may have a higher melting temperature than the second polymer.

The at least one discontinuous emitter band may form electrodes of a plurality of electrode pairs. The elongated tube may include a central lumen for accommodating a guide wire.

According to an aspect, a method of fabricating a catheter includes mounting a shock wave electrode assembly over a first tube, the shock wave electrode assembly comprising: at least one emitter band, the at least one emitter band forming a first electrode of an electrode pair, and at least one wire, a distal end of the at least one wire forming a second electrode of the electrode pair; sliding a second tube over the first tube and the shock wave electrode assembly such that the second tube at least partially surrounds the first tube and the shock wave electrode assembly; and applying heat to cause the second tube to adhere to the first tube such that at least a portion of the shock wave electrode assembly is at least partially embedded within the second tube.

The first tube may be an extruded polymer tube. The second tube may be an extruded polymer tube. The first tube may be formed of a first polymer, the second tube may be formed of a second polymer, and the first polymer may have a higher melting temperature than the second polymer.

The method may include removing a portion of the second tube to create a spark gap between the first electrode and the second electrode. The method may include removing a portion of the second tube such that an outer surface of at least a portion of the second tube aligns with an outer surface of the at least one emitter band. The method may include mounting an enclosure over the second tube such that the enclosure surrounds the shock wave electrode assembly.

According to an aspect, a catheter system for treating an occlusion in a body lumen includes a catheter comprising, at a distal end portion of the catheter: an inner catheter body, at least one shock wave electrode pair comprising at least one discontinuous emitter band having an enlarged central region and one or more leg regions extending from the enlarged central region, the one or more leg regions at least partially encircling a circumference of the inner catheter body, and a wire located adjacent the at least one discontinuous emitter band, an outer catheter body at least partially encapsulating the at least one discontinuous emitter band, and an enclosure in fluid communication with a fluid lumen of the catheter and surrounding the at least one shock wave electrode pair; and a power source electrically connected to the wire and configured to apply a voltage across the electrode pair to generate a shock wave within the enclosure.

The one or more leg regions may include a pair of leg regions, each extending from the enlarged region. Each of the pair of leg regions may have a leg end and the legends may not connect to each other. Each of the pair of leg regions may have a leg end and the leg ends may overlap each other. The leg regions of the pair of leg regions may be substantially the same in length or may have different lengths. At least one of the enlarged central region or one or more leg regions may include one or more slots for attaching one or more wires to the at least one discontinuous emitter band. The at least one discontinuous emitter band may include a pair of discontinuous emitter bands connected therebetween by a connecting region, and a width of the enlarged central region of at least one of the pair of discontinuous emitter bands is greater than a width of the connecting region. The connecting region may be fixedly joined to at least one of the one or more leg regions of each of the pair of discontinuous emitter bands.

According to an aspect, a catheter for treating an occlusion in a body lumen includes an elongated tube comprising an inner layer and an outer layer; a shock wave electrode assembly at least partially embedded within the elongated tube, the electrode assembly comprising: at least one emitter band comprising an enlarged region and an aperture extending through the enlarged region, an edge of the aperture of the at least one emitter band forming a first electrode of an electrode pair, and at least one wire, a distal end of the at least one wire forming a second electrode of the electrode pair such that the electrode pair emits a shock wave when a voltage pulse is applied across the electrode pair, wherein the at least one emitter band encircles the inner layer and the outer layer at least partially surrounds the at least one emitter band; and an enclosure surrounding the shock wave electrode assembly.

The enlarged region may be a first enlarged region and the at least one emitter band may further include a second enlarged region and at least one connecting region extending between the first enlarged region and the second enlarged region. A width of at least one of the first enlarged region or the second enlarged region may be greater than a width of the at least one connecting region. At least one of the first enlarged region or the second enlarged region may include one or more slots for attaching one or more wires to the at least one emitter band.

The aperture may have an elliptical shape. The outer layer may overlay at least a portion of an outer surface of the at least one emitter band. The inner layer may encapsulate at least a portion of the at least one wire. The inner layer may be formed of a first polymer, the outer layer may be formed of a second polymer, and the first polymer may have a higher melting temperature than the second polymer. The at least one emitter band may form electrodes of a plurality of electrode pairs.

According to an aspect, a catheter for treating an occlusion in a body lumen includes an elongated tube comprising an inner layer and an outer layer; a shock wave electrode assembly at least partially embedded within the elongated tube, the electrode assembly comprising: an emitter band that forms a first electrode of an electrode pair, and a wire including a distal end that forms a second electrode of the electrode pair, wherein the emitter band at least partially encircles the inner layer and the outer layer at least partially surrounds the emitter band, wherein the shock wave electrode assembly is configured to emit a shock wave when a voltage pulse is applied across the electrode pair; and an enclosure surrounding the shock wave electrode assembly.

The emitter band may be a discontinuous emitter band. The emitter band may include an inner surface that faces toward the inner layer, an outer surface that faces away from the inner layer, and an outer edge extending between the inner layer and the outer layer. The outer edge may be spaced apart from the distal end of the wire to define a spark gap sufficient to generate the shock wave. The emitter band may include a first enlarged region, a second enlarged region, and a connecting region extending between the first and second enlarged regions. The emitter band may include an aperture extending through one of the first enlarged region and second enlarged region. The aperture may be partially defined by an edge, the edge forming the first electrode and being spaced apart from the distal end of the wire to define a spark gap sufficient to generate the shock wave.

The emitter band may include a first discontinuous emitter band and a second discontinuous emitter band spaced longitudinally apart from the first discontinuous emitter band, the distal end of the wire may be positioned proximate to the first discontinuous emitter band, the wire may include a proximal end positioned proximate to the second discontinuous emitter band, the electrode pair may include a first electrode pair defined by the distal end of the wire and the first discontinuous emitter band and a second electrode pair defined by the proximal end of the wire and the second discontinuous emitter band, and the shock wave electrode assembly may be configured to emit a first shock wave when a voltage pulse is applied across the first electrode pair and a second shock wave when the voltage pulse is applied across the first electrode pair.

The emitter band may fully encircle the inner layer. The emitter band may include a first enlarged region, a second enlarged region, and a connecting region extending between the first and second enlarged regions. The emitter band may include an aperture extending through one of the first enlarged region and second enlarged region. The aperture may be partially defined by an edge, the edge forming the first electrode and being spaced apart from the distal end of the wire to define a spark gap sufficient to generate the shock wave.

According to an aspect, a system for treating an occlusion in a body lumen includes any of the catheters described above; and a voltage pulse generator electrically connected to the shock wave electrode assembly and configured for applying voltage pulses to the electrode pair to generate shock waves.

According to an aspect, a method of treating an occlusion in a body lumen includes introducing any of the catheters above into the body lumen; advancing the catheter through the body lumen to a position proximate to the occlusion; and applying voltage pulses across the electrode pair to generate shock waves inside the enclosure to treat the occlusion.

In some embodiments, any of the features of any of the embodiments described above and/or described elsewhere herein may be combined, in whole or in part, with one another. Additional advantages will be readily apparent to those skilled in the art from the following figures and detailed description. The aspects and descriptions herein are to be regarded as illustrative in nature and not restrictive.

### BRIEF DESCRIPTION OF THE FIGURES

Illustrative aspects of the present disclosure are described in detail below with reference to the following figures. It is intended that the embodiments and figures disclosed herein are to be considered illustrative rather than restrictive.

FIG. 1 illustrates an exemplary system comprising a catheter and a voltage pulse generator, with the catheter pictured treating a stenosis in a blood body lumen, according to one or more aspects of the present disclosure.

FIG. 2 illustrates a perspective view of the distal end of an exemplary catheter, according to one or more aspects of the present disclosure.

FIG. 3 illustrates an exploded view of the distal end of an exemplary catheter, according to one or more aspects of the present disclosure.

FIGS. 4A-4B illustrate cross-sectional views of the distal end of exemplary catheters, according to one or more aspects of the present disclosure. FIG. 4A illustrates an exemplary catheter having an emitter band at least partially embedded in the outer layer of an elongated tube. FIG. 4B illustrates an exemplary catheter having an emitter band at least partially encapsulated within the outer layer of an elongated tube.

FIGS. 5A and 5B illustrate exemplary emitter bands that may be included in a catheter, according to one or more aspects of the present disclosure.

FIG. 6 illustrates an exemplary shock wave electrode assembly including a plurality of the exemplary emitter bands shown in FIG. 5A, according to one or more aspects of the present disclosure.

FIG. 7 illustrates an exemplary emitter band that may be included in a catheter, according to one or more aspects of the present disclosure.

FIG. 8 illustrates an exemplary shock wave electrode assembly including a plurality of the exemplary emitter bands shown in FIG. 7, according to one or more aspects of the present disclosure.

FIGS. 9A-9C illustrate various exemplary emitter bands that may be included in a catheter, according to one or more aspects of the present disclosure.

FIG. 10 illustrates an exemplary emitter band soldered to two wires, according to one or more aspects of the present disclosure.

FIG. 11 illustrates a flow chart of an exemplary method of treating an occlusion in a body lumen, according to one or more aspects of the present disclosure.

FIG. 12 illustrates a flow chart of an exemplary method of fabricating a catheter, according to one or more aspects of the present disclosure.

FIGS. 13A and 13B illustrate a perspective view and a side view, respectively, of an exemplary emitter band according to one or more aspects of the present disclosure.

FIGS. 14A and 14B illustrate an exemplary emitter band, according to one or more aspects of the present disclosure. FIG. 14A illustrates the arrangement of the emitter band with a pair of wires and FIG. 14B illustrates the emitter band alone.

FIGS. 15A-15C illustrate an exemplary emitter assembly according to one or more aspects of the present disclosure.

FIGS. 16A-16C illustrate an exemplary emitter band that may be included in a catheter, according to one or more aspects of the present disclosure.

FIG. 16D illustrates a cross-sectional view of an exemplary catheter including the exemplary emitter band shown in FIGS. 16A-16C, according to one or more aspects of the present disclosure.

FIGS. 17A-17E illustrate an exemplary emitter band that may be included in a catheter, according to one or more aspects of the present disclosure.

FIG. 17F illustrates an exemplary shock wave electrode assembly including a plurality of the exemplary emitter bands shown in FIGS. 17A-17E, according to one or more aspects of the present disclosure.

FIG. 18A illustrates an exemplary emitter band that may be included in a catheter, according to one or more aspects of the present disclosure.

FIG. 18B illustrates an exemplary shock wave electrode assembly including a plurality of the exemplary emitter bands shown in FIG. 18A, according to one or more aspects of the present disclosure.

### DETAILED DESCRIPTION

The following description is presented to enable a person of ordinary skill in the art to make and use the various embodiments and aspects thereof disclosed herein. Descriptions of specific catheters, systems, methods, and applications are provided only as examples. Various modifications to the examples described herein will be readily apparent to those of ordinary skill in the art, and the general principles described herein may be applied to other examples and applications without departing from the spirit and scope of the various embodiments and aspects thereof. Thus, the various embodiments and aspects thereof are not intended to be limited to the examples described herein and shown but are to be accorded the scope consistent with the claims.

Described herein are shock wave catheters that include at least one discontinuous emitter band that forms an electrode of a shock wave electrode assembly. The discontinuous structure of the emitter band allows the band to be compressed to a smaller diameter during assembly. For example, the discontinuous emitter band may be mounted onto one or more layers of the catheter body in such a way that the discontinuous band is smaller in diameter than in its unmounted state. Such a catheter may be fabricated by mounting components of an electrode assembly, including at least one discontinuous emitter band, on a first tube that forms an inner layer of the catheter body. The discontinuous band can then be compressed to snugly fit or be embedded within the catheter body. For example, a second tube forming an outer layer of the catheter body may be positioned over the inner layer and electrode assembly exposed to an external stimulus (e.g., heat, light, magnetic field, or a solution), such that the outer layer shrinks down onto the inner layer and electrode assembly, compressing the discontinuous emitter band into a smaller diameter state.

Advantageously, the use of a discontinuous emitter band (or bands) allows for the diameter of the band to be changed (e.g., enlarged and/or reduced) during the fabrication process, such that a single-sized band may be compatible with a wide variety of catheters having a range of catheter body diameters. Additionally, the size of the discontinuous emitter bands may be reduced during steps of the fabrication process to reduce the overall crossing profile of the resulting catheter. For instance, the emitter band can be mounted over the inner layer tube in a first relatively larger diameter state, and the band can then be compressed to a reduced-diameter state during the shrinking of the second tube over the first tube. Accordingly, the fabrication process can produce a catheter having at least one emitter band that is at least partially embedded within the catheter body in the reduced-diameter state. Such catheters may have a relatively lower crossing profile compared to catheters fabricated by stacking the electrodes and other components around the outer surface of the catheter body. The discontinuous form of the emitter band may be further advantageous in being more flexible than conventional continuous bands, leading to a more flexible catheter. Such discontinuous emitter bands may further be used with catheters having a variable or tapered diameter in the operational region. Such catheter designs allow the catheters to maneuver tighter regions of vasculature and treat more severely occluded regions of a vessel.

Also described herein are systems including the catheters described above, as well as methods for using the catheters and methods of fabricating the catheters.

As used herein, the term “electrode” refers to an electrically conducting element (typically made of metal) that receives electrical current and subsequently releases the electrical current to another electrically conducting element. In the context of the present disclosure, electrodes are often positioned relative to each other, such as in an arrangement of an inner electrode and an outer electrode. Accordingly, as used herein, the term “electrode pair” refers to two electrodes that are positioned adjacent to each other such that application of a sufficiently high voltage to the electrode pair will cause an electrical current to transmit across the gap (also referred to as a “spark gap”) between the two electrodes (e.g., from an inner electrode to an outer electrode, or vice versa, with the electricity passing through a conductive fluid therebetween). In the context of the present disclosure, the term “emitter” broadly refers to the region of an electrode assembly where the current transmits across one or more electrode pairs, generating a shock wave. In some contexts, one or more electrode pairs may also be arranged in and referred to as an electrode assembly. In the context of the present disclosure, the term “electrode assembly” or “shock wave electrode assembly” broadly refers to the wires, electrodes, and other components of the catheter used to transmit current from a voltage source across one or more electrode pairs to generate shock waves. One or more of the electrode pairs in an electrode assembly can be wired in series to generate shock waves together. In some instances, electrode pairs can be wired on separate circuits or separate circuit branches to be operated separately. Accordingly, during a shock wave treatment, shock waves may be generated at all of the electrode pairs of the electrode assembly, or at only a particular subset of the electrode pairs.

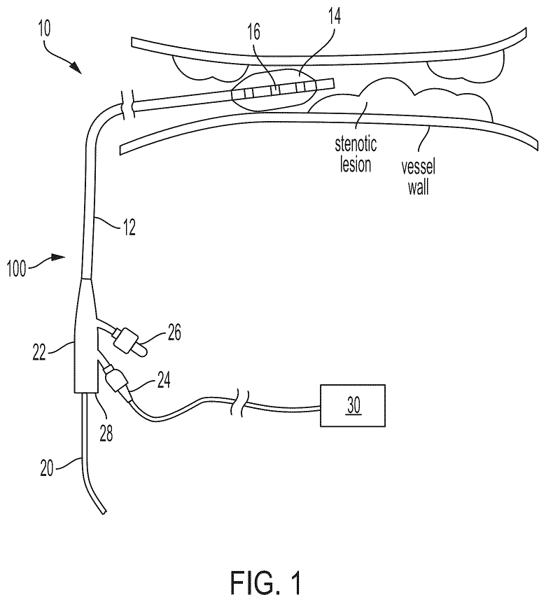

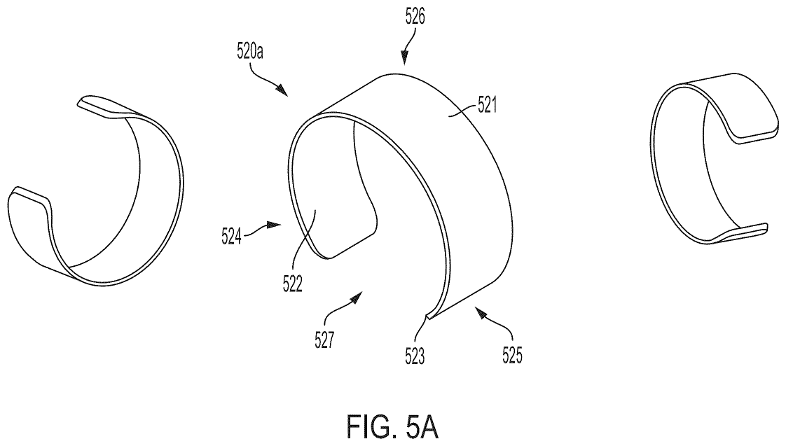

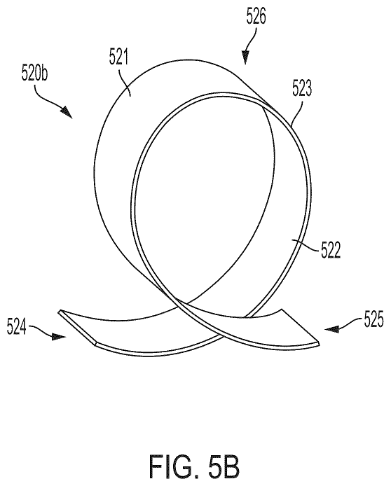

As used herein, the term “emitter band” refers to a region of conductive material that at least partially encircles a portion of the catheter and may form one or more electrodes of one or more electrode pairs of an electrode assembly. An emitter band may form a location of one or more emitters. An emitter band may be generally ring-shaped and/or cylindrical such that it can be mounted over a tubular catheter body (e.g., the elongated tube 12 of FIG. 1). As described further below, a “discontinuous emitter band” refers to an emitter band that extends circumferentially around a region of the catheter and includes two ends that are not joined together. For instance, a discontinuous emitter band could be generally ring-shaped and/or cylindrical with a split at a location along the circumference of the band that forms a gap between a first end and a second end of the band. In other words, a discontinuous band will have unconnected ends or legs, which may optionally be aligned (e.g., almost a ring) or non-aligned (e.g., a coil shape) around a central radial point of the discontinuous band. Such unconnected ends or legs may be configured to wrap around an inner layer of the catheter, thereby increasing the securement strength. A connecting region between the first end and the second end encircles a portion of a catheter. In some examples, a discontinuous emitter band encircles only a portion of the circumference of the catheter. For instance, the band may be crescent-shaped or C-shaped in profile and have a connecting region that encircles less than 360 degrees of a region of the catheter (e.g., 90°, 120°, 150°, 180°, 210°, 240°, 270°, 300°, and/or) 330°. In another example, a discontinuous emitter band could wind around a region of the catheter one or more times, with a connecting region that encircles more than 360 degrees (e.g., 390°, 450°, and/or 540°). of the circumference of the region of the catheter; in such an example, the unconnected ends may overlap. Similarly, a discontinuous emitter band could wind around a region of the catheter one time, encircling 360 degrees of the circumference of a lumen region, where the ends or legs of the discontinuous emitter band are formed to not align with each other. In such examples, the discontinuous emitter band may be generally keyring- or spiral-shaped. Various exemplary discontinuous emitter bands are shown in FIGS. 5A-5B, FIG. 7, FIGS. 9A-9C, FIGS. 13A-13B, and FIGS. 14A-14B and described below.

As provided herein, it should be appreciated that any disclosure of a numerical range describing dimensions or measurements such as thicknesses, length, weight, time, frequency, temperature, voltage, current, and/or angle is inclusive of any numerical increment or gradient within the ranges set forth relative to the given dimension or measurement. Furthermore, numerical designators such as “first,” “second,” “third,” and/or “fourth” are merely descriptive and do not indicate a relative order, location, or identity of elements or features described by the designators. For instance, a “first” shock wave may be immediately succeeded by a “third” shock wave, which is then succeeded by a “second” shock wave. As another example, a “third” emitter band may be placed proximate to a “a “second” wire and be used to generate a “first” shock wave and vice versa. Accordingly, numerical designators of various elements and features are not intended to limit the disclosure and may be modified and interchanged.

In the following description of the various embodiments, it is to be understood that the singular forms “a,” “an,” and “the” used in the following description are intended to include the plural forms as well, unless the context clearly indicates otherwise. It is also to be understood that the term “and/or” as used herein refers to and encompasses any and all possible combinations of one or more of the associated listed terms. It is further to be understood that the terms “includes,” “including,” “comprises,” and/or “comprising,” when used herein, specify the presence of stated features, integers, steps, operations, elements, components, and/or units but do not preclude the presence or addition of one or more other features, integers, steps, operations, elements, components, units, and/or groups thereof.

FIG. 1 depicts an exemplary system 10 for treating a lesion in a body lumen, such as the stenotic lesion in the vessel wall pictured in FIG. 1. The system 10 includes a shock wave catheter 100 and a voltage source 30, according to one or more examples. The shock wave catheter 100 includes an elongated tube 12 and at least one enclosure 14 (e.g., an inflatable angioplasty balloon or a non-inflatable cap) enclosing one or more emitter(s) 16 for generating shock waves. The system 10 also includes a voltage source 30 that is electrically connected to and configured for applying a voltage (e.g., a high-voltage pulse or a series of high-voltage pulses) to one or more of the emitters 16. When the distal end of the catheter 100 has been positioned in a body lumen (e.g. the vessel wall of FIG. 1), voltage pulses can be applied by the voltage source 30 to one or more of the emitters 16 to create shock waves inside the enclosure(s) 14. When a voltage pulse is delivered by the voltage source 30, current flows across a spark gap between an electrode pair of the emitter(s) 16 to generate shock waves at the spark gap. The shock waves propagate generally outwardly from the emitter(s) 16 and toward the inner surface of the enclosure 14, through the material of the enclosure, and into a lesion in a body lumen proximate to the enclosure where the energy breaks apart hardened plaque. In various examples, the lesion could be a calcified region of vasculature, a thrombus or an occlusion in vasculature, arteriosclerotic plaque, or a lesion in some other body lumen, such as a kidney stone in a ureter.

The elongated tube 12 of the catheter 100 extends distally from a handle 22 of the catheter and includes the emitter(s) 16 and the enclosure(s) 14 mounted proximate to its distal end. As described in more detail below, the distal end of the elongated tube 12 may be formed from one or more compliant polymeric materials and is configured to be inserted into a body lumen of a patient, such as a blood vessel, a valve, a ureter, or some other body lumen. The elongated tube 12 may include various lumens and/or channels sized for carrying fluid, conductive wires, and other components of the catheter 100 between its proximal handle 22 and the enclosure 14, such as a fluid lumen for carrying fluid introduced through a fluid port 26 and various conductive wires that enter through one or more wire ports 24. A fluid lumen of the catheter may be in fluid communication with the enclosure 14. In some examples, the handle 22 is configured to receive a guide wire 20 through a guide wire port 28. In such examples, a guide wire 20 may be inserted into the guide wire port 28 and extended through a guide wire lumen of the elongated tube 12 to aid in insertion and positioning of the enclosure(s) 14 proximate to a lesion in a body lumen. However, in some examples, the elongated tube 12 does not include a guide wire lumen and the handle 22 does not include a guide wire port 28.

The enclosure(s) 14 of the catheter 100 may extend circumferentially around a portion of the elongated tube 12 and be sealed to a region of the elongated tube near the distal end of the tube. In some examples, the enclosure(s) 14 surrounds one or more of the emitter(s) 16, such that the emitter(s) 16 generate shock waves inside the enclosure(s). The enclosure(s) 14 can be filled with a conductive fluid, such as saline, that conducts the shock waves and allows the shock waves to propagate outwardly from the emitter(s) 16 and toward the walls of the enclosure(s). In some examples, the conductive fluid may also contain an x-ray contrast fluid to permit fluoroscopic viewing of the catheter 100 and enclosure 14 by a surgeon during use. In some examples, when the catheter 100 is positioned in situ, the enclosure(s) 14 can be inflated (e.g., with the conductive fluid) such that the enclosure expands (i.e., inflates) to contact a body lumen (e.g., the walls of an artery proximal to a calcified lesion, such as the stenotic lesion shown in FIG. 1). When inflated, the enclosure(s) 14 provides an annular channel around the elongated tube 12 and creates a space between the emitter(s) 16 and the walls of the enclosure(s) 14 to minimize the risk of rupturing the enclosure(s) 14 during a shock wave treatment. In a deflated state, the enclosure(s) 14 may be positioned closely proximate to the elongated tube 12 and optionally in a folded state, which improves the maneuverability of the catheter 100 during insertion and positioning. In some examples, the enclosure(s) 14 is formed from a compliant material, such as a compliant polymer. In a particular example, the enclosure 14 is an inflatable angioplasty balloon. However, in other examples, the enclosure 14 is not inflatable and may be formed from a material that can be pressurized without inflating.

To operate the catheter 100, a physician optionally inserts a guide wire 20 into a body lumen. The physician can track the position of the guide wire 20 and catheter 100 within a patient by use of real-time and/or static imaging devices, including x-ray imaging, intravascular ultrasound (IVUS), optical coherence tomography (OCT), and other such techniques. The physician then positions the elongated tube 12 over the proximal end of the guide wire such that the guide wire extends through the elongated tube 12 and uses the guide wire 20 to guide the elongated tube 12 into position proximate to a lesion in a body lumen. Once positioned, the enclosure 14 can be filled with a conductive fluid through the fill port(s) 26, optionally such that the enclosure inflates to contact the wall of the body lumen. The voltage source 30 is then used to deliver one or more high voltage pulses to the emitter(s) 16 to create one or more shock waves within the enclosure 14 and within the body lumen being treated.

In some examples, the magnitude of the shock waves can be controlled by controlling the magnitude of the pulsed voltage, the current, the duration, and the repetition rate of the voltage supplied by the voltage source 30. Furthermore, in examples where one or more emitter(s) 16 are wired on separate circuits or separate circuit branches to be operated separately, a user of the catheter 100 may selectively emit shock waves at only a particular subset of emitters of the catheter by applying voltage pulses to generate shock waves at only that subset of the emitters. The physician may start with low energy shock waves and increase the energy as needed to disrupt the lesion and crack calcified plaques. In some examples, a physician may first generate shock waves at a first subset of the emitters 16 (e.g., a distal subset of emitters) and may continue treatment by generating shock waves at a second subset of emitters (e.g., a central or proximal subset of emitters). Repeated shock waves can be delivered, and the catheter 100 can be repositioned or advanced further in the body lumen to continue treatment. When the shock wave treatment is completed, the enclosure(s) 14 can be deflated and the distal end of the catheter 100 removed from the body lumen.

As described in further detail below, each emitter 16 may be formed from one or more regions of conductive material (e.g., electrodes) that are spaced apart by a spark gap to form one or more electrode pairs. For instance, an exemplary emitter 16 may be formed from a conductive emitter band that is mounted around the elongated tube 12 and one or more conductive regions of a wire or wires (e.g., an un-insulated portion of the wire(s) or an exposed end of the wire(s)) that are spaced apart from the emitter band. In a particular example, each emitter(s) 16 is formed from an emitter band and the conductive regions of two wires placed in close proximity to the emitter band, such that when a voltage is applied across the wires, current flows down a first wire, across the emitter band, and down the second wire, generating shock waves at a first spark gap between the first wire and the emitter band a second spark gap between the emitter band and the second wire. Accordingly, each emitter 16 may include one or more electrode pairs and may be configured for generating one or more shock waves. In some examples, the emitter(s) 16 includes a first electrode pair on a first side of the emitter and a second electrode pair on a second side of the emitter, such that shock waves can be generated in two or more directions relative to the elongated tube 12 to treat a larger area of a lesion.

Any desired number of emitters may be provided in an exemplary shock wave catheter, such as the catheter 100 of FIG. 1. For instance, catheter 100 could include one, two, three, four, five, six, eight, or more than eight emitters. The emitter(s) 16 of catheter 100 may be arranged in any desired configuration along the distal end of the elongated tube 12. For instance, in some examples the emitter(s) 16 are arranged on the elongated tube 12 as a proximal subset of emitters and a distal subset of emitters. In other examples, the emitter(s) 16 may be spaced evenly along a length of the elongated tube 12.

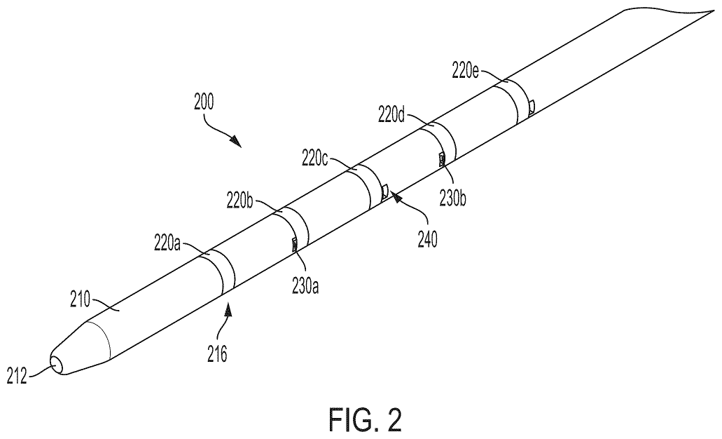

FIG. 2 illustrates the distal end of an exemplary catheter 200 that includes at least one emitter 216 formed from an emitter band 220a that is mounted circumferentially around an elongated tube 210. The catheter 200 could be included in a system for treating lesions in vasculature, such as the catheter 100 of system 10 of FIG. 1. In some examples, the catheter 200 includes a plurality of emitter bands 220a-220e mounted on the elongated tube 210 and spaced longitudinally from one another along a length of the catheter 200. For example, the catheter 200 of FIG. 2 is pictured as having five emitter bands 220a, 220b, 220c, 220d, 220e spaced at equal distances along the distal end of the catheter 200. However, a shock wave catheter could include any number of emitter bands arranged in any desired configuration.

To reduce the crossing profile of the catheter 200, one or more of the emitter bands 220a-220e may be at least partially recessed (e.g., embedded) into the material of the elongated tube 210. Embedding at least a portion of an emitter band 220a (or bands) in the material of the elongated tube 210 may result in a reduced crossing profile of the catheter's distal end compared to standard catheters having emitter bands and other components mounted on top of and projecting away from the surface of the catheter body. Such a configuration may improve the catheter's maneuverability and allow the catheter 200 to treat tighter and more severely occluded regions of vasculature.

The elongated tube 210 of the catheter body may be formed from a compliant polymeric material that allows the elongated tube 210 to bend and flex when maneuvered through a body lumen. For instance, in some examples the elongated tube 210 is formed from PTFE (Teflon), polyether block amide (e.g., Pebax), nylon, urethane, or some other polymeric material. The elongated tube 210 includes a central lumen 212, which can accommodate a guide wire for facilitating the insertion and advancement of the distal end of the catheter through a body lumen. In some examples, the distal tip of the elongated tube 210 is formed (e.g., machined or otherwise manipulated) into a taper to facilitate advancement of the catheter 200 while reducing the risk of injury to a body lumen.

As mentioned above, in some examples one or more of the emitter bands 220a-220e is at least partially embedded within the material of the elongated tube 210. For instance, a portion of the emitter band 220a may be embedded in the material of the elongated tube 210, while a second portion of the emitter band 220a is not embedded in the material of the elongated tube 210. In some examples, at least a portion of one or more of the emitter bands 220a-220e is surrounded by or encapsulated within the material of the elongated tube 210. For instance, a first portion (e.g., an end or an attachment region) of the emitter band 220a may be encapsulated within the material of the elongated tube 210, while a second portion of the emitter band 220a is exposed (i.e., is not encapsulated). In some examples, the emitter band 220a is embedded or encapsulated such that the outer diameter of the emitter band 220a corresponds to (e.g., is flush with) the outer diameter of the elongated tube 210 and the emitter band 220a does not project outwardly from the catheter body. Alternatively, the emitter band 220a can be embedded into the elongated tube 210 such that the outer diameter of the band is recessed relative to the outer diameter of the elongated tube 210. The embedding or encapsulation of the emitter bands 220a-220e may improve the attachment of the bands to the elongated tube 210 and further reduce the crossing profile of the catheter body.

A portion of the emitter band 220a forms a first electrode of an electrode pair. As mentioned above, in some examples a second electrode of an electrode pair is formed from a conductive portion of a wire (e.g., a distal or a proximal end of the wires 230a and/or 230b). For instance, one or more wires 230a-230b may extend along the length of the elongated tube 210 and are optionally at least partially encapsulated within the material of the elongated tube. For instance, in the example illustrated in FIG. 2, the ends of the wires 230a-230b are not encapsulated, while the remainder of the wires are encapsulated within the material of the elongated tube 210. The ends of the wires 230a-230b are positioned in proximity to the emitter bands 220b and/or 220d, such that when a voltage is applied to the wires 230a-230b, current can flow across spark gaps between the conductive ends of the wires and one or more of the emitter bands to generate shock waves. For instance, in the catheter 200 shown in FIG. 2, shock waves can be formed in at least five locations along the catheter's distal end corresponding to the locations between the emitter bands 220a-220e and the ends of wires (e.g., wires 230a-230b and/or other wires not pictured) positioned proximate to the emitter bands.

In some examples, the catheter 200 may be shaped or modified by removing at least a portion of the material of the elongated tube 210. For instance, a portion of the material may be removed at a spark gap between an emitter band and a wire to create one or more recesses 240 (e.g., one or more holes) in the elongated tube 210. The recesses allow current to flow between one or more of the emitter bands 220a-220e and the wires 230a-320b and may also direct the shock waves formed at the spark gaps to propagate in an outwardly from the catheter body. In some examples, a hole in the outer layer provides the spark gap between the first electrode and the second electrode of the electrode pair. In some examples, a portion of the material of the elongated tube 210 is removed to reduce the crossing profile of the catheter 200 (e.g., by removing material until the outer diameter of the elongated tube 210 is flush with the outer diameter of one or more of the emitter bands 220a-220e) and/or to shape the distal tip of the catheter 200.

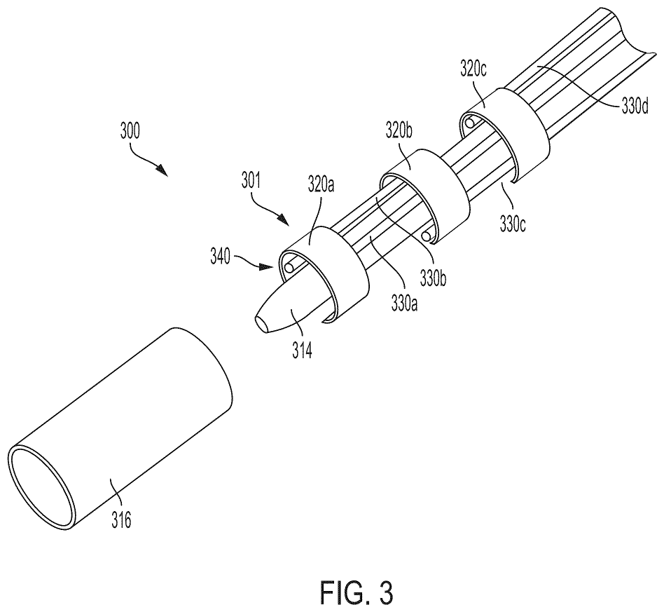

An exemplary shock wave catheter may be assembled in a stepwise process that includes mounting a second tube over a first tube to form a multi-layer elongated tube with one or more emitter band(s), wire(s), and/or other components embedded or encapsulated in the layers of the tube. For instance, FIG. 3 illustrates an exploded view of the distal end of a shock wave catheter 300, such as either of the catheters 100 and 200 shown in respective FIG. 1 and FIG. 2, prior to assembly of the catheter. The body of catheter 300 is formed from a first tube 314 and a second tube 316 that can be sealed together to form an elongated tube (e.g., the elongated tube 210 of catheter 200 of FIG. 2). The first tube 314 may be an elongate tube. A shock wave electrode assembly can be mounted over the first tube 314 and can be adhered within the catheter body by sliding a second tube 316 over the first tube and shrinking the second tube over the first tube, e.g., by heating or otherwise processing the second tube to shrink the second tube over the first tube. In some examples, when the catheter 300 has been assembled, the first tube 314 forms an inner layer of the catheter body (e.g., an inner catheter body), and the second tube 316 forms an outer layer of the catheter body (e.g., an outer catheter body). The multi-layer configuration of the catheter body may simplify the fabrication of catheters, and particularly the fabrication of reduced-diameter catheters having various components (e.g., a shock wave electrode assembly) embedded within the catheter body.

An exemplary shock wave electrode assembly 301 of a catheter 300 includes one or more emitter bands, such as the first 320a, second 320b, and third 320c emitter bands pictured in FIG. 3. In some examples, one or more of the emitter bands 320a-320c is discontinuous and configured to encircle only a portion of the circumference of the first tube 314. As described above, the first tube and the second tube may be sealed together to form an elongated tube, with the first tube forming an inner layer of the catheter body. Thus, emitter bands 320a-320c may partially encircle this inner layer of the catheter body or elongated tube. However, in other examples, one or more of the emitter bands 320a-320c are discontinuous and configured to encircle the entire circumference of the first tube 314 one or more times (e.g., in a coil shape). In various examples, an emitter band is made a material and shaped to be radially flexible. The discontinuous configuration of an emitter band may allow the diameter of the band to be controlled by compressing or expanding the band into a relatively larger- or smaller-diameter configuration. By creating two free ends, the discontinuity in a discontinuous emitter band permits the band to flex and change shape during use. An electrode assembly including a discontinuous emitter band may have increased radial flexibility compared to emitter assemblies featuring continuous ring emitter bands, thereby facilitating the fabrication of catheters including at least one discontinuous emitter band and providing improved navigability during use.

The shock wave electrode assembly also includes one or more conductive wires that extend longitudinally along the length of the first tube 314, such as the first 330a, second 330b, third 330c, and fourth 330d wires pictured in FIG. 3. Distal and/or proximal ends of the wires 330a-330d can be placed proximate to one or more of the emitter bands 320a-320c and spaced apart from the bands by a spark gap (e.g., spark gap 340) across which current can flow to generate shock waves.

In some examples, the electrode assembly is assembled on top of the first tube 314 during assembly of the catheter 300. For instance, the various wires 330a-330d can be arranged such that they extend longitudinally along the first tube 314, and the emitter bands 320a-320c can be slid loosely over the wires and the first tube 314. In some examples, the first tube 314 includes grooves, indentations, or other attachment structures, and the electrode assembly can be mounted on the first tube by positioning the wires 330a-330d within the grooves and sliding the emitter band(s) 320a-320c over the wires and the first tube. In such examples, the attachment structures or grooves may be formed by removing material from (e.g., ablating) the first tube 314 prior to assembling the catheter 300. In some examples, the surface of the first tube 314 is etched to improve the adhesion of the second tube 316 to the first tube.

The one or more emitter band(s) 320a-320c can then be compressed onto the first tube 314 to reduce the crossing profile of the electrode assembly. For instance, in some examples, an emitter band of the assembly has a first (e.g., unassembled) configuration having a first diameter, the first diameter being greater than the diameter of the first tube 314. For instance, the first diameter of an emitter band could be between approximately two hundredths of an inch (0.02 inches) and five hundredths of an inch (0.05 inches) or between approximately three hundreds of an inch (0.03 inches) and four hundredths of an inch (0.04 inches). Once the one or more emitter bands 320a-320c are mounted over the first tube 314, the emitter band(s) can be compressed into a second (e.g., assembled) configuration having a second diameter less than the first diameter of an emitter band in the first configuration. For instance, the second diameter of an emitter band could be between approximately one hundredth of an inch (0.01 inches) and three hundredths of an inch (0.03 inches) or between approximately two hundredths of an inch (0.02 inches) and three hundredths of an inch (0.03 inches). In some examples, the diameters of the emitter bands 320a-320c may be compressed to a second diameter that no less than 5%, no less than 10%, or no less than 15% smaller than a first diameter of the emitter band. In some examples, the emitter bands 320a-320c can be compressed to a second diameter that is up to 25% smaller compared to a first diameter. The relative amount of compression may be tailored to fit a desired size or catheter profile. For instance, a number of emitter bands having the same first diameter (e.g., the same diameter in an unassembled state) may be compressed to different second diameters that match the size or crossing profile of an assembled catheter. In some examples, the diameter of the emitter bands 320a-320c is compressed manually or by hand when the electrode assembly is mounted over the first tube 314 of the elongated tube. A crimping tool may be used to compress the one or more discontinuous emitter band to the assembled diameter. Additionally or alternatively, the diameter of the emitter bands 320a-320c may be compressed when the second tube 316 is sealed over the first tube 314. For instance, the emitter bands 320a-320c may be compressed into a smaller-diameter configuration by the inward force of the second tube 316 when the second tube shrinks over the first tube 314, e.g., by heat sealing. In various embodiments, compressing an emitter band to its second (e.g., final or assembled) diameter effectively embeds the band (and attached wires) into a portion of the catheter body.

In some examples, sealing the second tube 316 to the first tube causes the emitter bands 320a-320c and/or wires 330a-330d of the electrode assembly to be at least partially encapsulated by the second tube. For instance, in some examples, sealing the second tube 316 to the first tube 314 causes the second tube to encapsulate at least a portion of the emitter bands 320a-320c and/or wires 330a-330d of the electrode assembly. In other examples, sealing the second tube 316 to the first tube 314 completely encapsulates the wires 330a-330d and emitter bands 320a-320c of the assembly. In some examples, after sealing the second tube 316 to the first tube 314, material is removed from the second tube, e.g., to reveal a portion of the emitter bands 320a-320c and/or to reveal the spark gaps between the emitter bands and the wires 330a-330d to allow the shock waves to propagate outwardly from the elongated tube. The material may be removed, e.g., by laser ablation or with manual tools. In some examples, when the catheter 300 is assembled, the second tube 316 surrounds at least a portion of one or more of the emitter bands 320a-320. In some examples, a portion of each of the emitter bands is not surrounded by the second tube. For instance, in some examples, the second tube 316 does not surround a spark gap between a first electrode and a second electrode of an electrode pair (e.g., spark gap 340 between emitter band 320a and wire 330b).

Each of the first tube 314 and second tube 316 is shaped as a hollow tube and may be fabricated as an extruded polymer tube. In some examples, each of the first 314 and second 316 tubes is formed from a compliant polymeric material, such as such as a polytetrafluoroethylene (PTFE) (e.g., Teflon), PEBA (e.g., Pebax), nylon, urethane, or some other polymeric material. In some examples, the first tube 314 and second tube 316 are formed from the same polymeric material. However, in other examples, the first tube 314 is formed of a first polymeric material, the second tube 316 is formed of a second polymeric material different from the first polymeric material. For instance, to facilitate the heat sealing of the second tube 316 over the first tube 314, in some examples the first polymeric material has a higher melting temperature than the second polymeric material. In such examples, the heat-sealing of the second tube 316 over the first tube 314 does not deform or change the diameter of the first tube 314. In a particular example, the first tube 314 is formed from a fluoropolymer (e.g., PTFE), while the second tube 316 is formed from a PEBA material.

The second tube 316 has an inner diameter greater than the outer diameter of the first tube 314 and the electrode assembly, such that the second tube 316 can be loosely mounted over the first tube 314 and electrode assembly during assembly of the catheter 300. In some examples, the outer diameter of the first tube 314 is approximately twenty-three thousandths of an inch (0.023 inches). However, in some examples the outer diameter may be greater or lesser, for instance, between two hundredths of an inch (0.02 inches) and three hundredths of an inch (0.03 inches), between two hundredths of an inch (0.02 inches) and twenty-five thousandths of an inch (0.025 inches), between twenty-two thousandths of an inch (0.022 inches) and twenty-four thousandths of an inch (0.024 inches), greater than two hundredths of an inch (0.02 inches), or less than twenty-five thousandths of an inch (0.025 inches). In some examples, the inner diameter of the second tube 316 is approximately twenty-eight thousandths of an inch (0.028 inches) prior to adhering the second tube to the first tube 314. However, in some examples the inner diameter may be greater or lesser, for instance, between two hundredths of an inch (0.02 inches) and three hundredths of an inch (0.03 inches), between twenty five thousandths of an inch (0.025 inches) and three hundredths of an inch (0.03 inches), between twenty-seven thousandths of an inch (0.027 inches) and twenty-nine thousandths of an inch (0.029 inches), greater than twenty-five thousandths of an inch (0.025 inches), or less than three hundredths of an inch (0.3 inches). In some examples, the sealing of the second tube 316 to the first tube 314, e.g., by heating the second tube, decreases the diameter of the second tube such that the inner diameter of the second tube is equal to the outer diameter of the first tube.

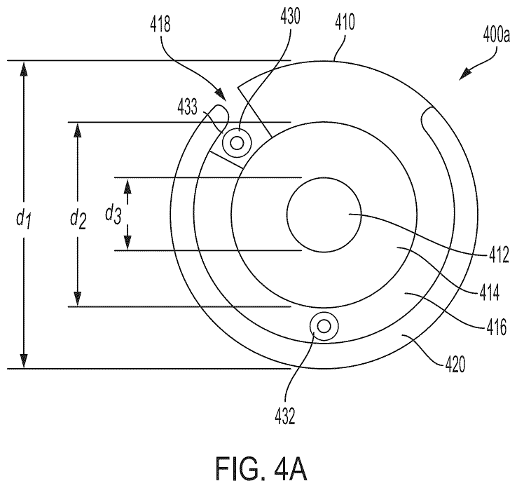

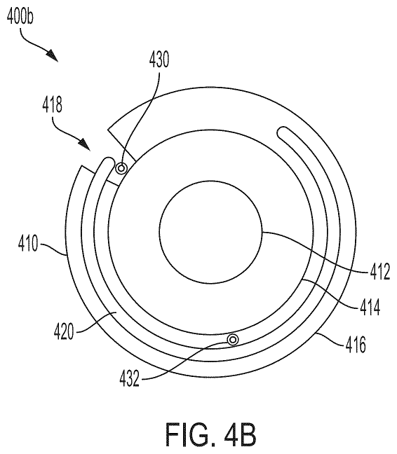

When the second tube 316 is sealed to the first tube 314, the resulting catheter may include a multi-layer elongated tube having emitter band(s), wire(s) and other components embedded within or encapsulated by the layers of the tube. For instance, FIGS. 4A-4B illustrate cross-sectional views of the distal end of exemplary catheters 400a-400b showing a multi-layered elongated tube 410 formed according to the exemplary method described above with respect to FIG. 3. The exemplary catheters 400a-400b can be used as any of the catheters 100, 200, and 300 shown in FIGS. 1-3 and described supra. FIG. 4A illustrates an example of a catheter 400a that includes an emitter band 420 that is embedded within the layers of the elongated tube 410 and at least partially surrounded by the material of the elongated tube. FIG. 4B illustrates an example of a catheter 400b including an emitter band 420 that is at least partially encapsulated by the material of the elongated tube 410. The catheters 400a-400b pictured in FIGS. 4A-4B include at least an emitter band 420, a first wire 430, and a second wire 432. However, additional wires and emitter bands may be present and not visible in the cross-sections shown in FIGS. 4A-4B.

As described above, the body of a catheter may be formed as an elongated tube 410 that includes one or more layers, such as an inner layer 414 and an outer layer 416. As described in relation to FIG. 3, the inner layer 414 and outer layer 416 may be formed from one or more tubes that are sealed together during the fabrication of the catheter. For instance, the inner layer 414 may be formed from a first tube (e.g., the first tube 314 of FIG. 3), and the outer layer 416 may be formed from a second tube (e.g., the second tube 316 of FIG. 3) that is shrunk over the first tube. When the catheter 400a-400b is assembled, the inner layer 414 and outer layer 416 may be generally concentric, with the outer surface of the inner layer 414 or first tube at least partially abutting the inner surface of the outer layer 416 or second tube after assembly of the catheter. In some examples, the inner surface of the emitter band 420 directly abuts the inner layer 414. For instance, in examples where the wires 430, 432 are mounted within grooves formed in the inner layer 414, the emitter band 420 may be mounted over the wires and directly abutting the outer surface of the inner layer. However, in other examples and as seen in FIGS. 4A-4B, at least a portion of the outer layer 416 is between the inner surface of the emitter band 420 and the outer surface of the inner layer 414.

The inner layer 414 of the elongated tube 410 includes an outer diameter (e.g., d2) and an inner diameter (e.g., d3), the inner diameter forming the outer diameter of a central lumen 412 of the elongated tube. In some examples, the central lumen 412 is for accommodating a guide wire, and the central lumen may be sized to loosely receive a guide wire (e.g., with the diameter of the central lumen corresponding to the diameter of a guide wire). In some examples, the central lumen 412 is sized to receive a guide wire having a diameter of fourteen thousandths of an inch (0.014 inches). For instance, the inner diameter of the inner layer 414 could be approximately fifteen thousandths of an inch (0.015 inches). However, in some examples, the inner diameter of the inner layer may be greater or lesser, for instance, between one hundredth of an inch (0.01 inches) and two hundredths of an inch (0.02 inches), between fifteen thousandths of an inch (0.015 inches) and two hundredths of an inch (0.02 inches), between fourteen thousandths of an inch (0.014 inches) and sixteen thousandths of an inch (0.016 inches), or greater than fourteen thousandths of an inch (0.014 inches). In some examples, the outer diameter of the inner layer 414 is approximately twenty-three thousandths of an inch (0.023 inches). However, in some examples the outer diameter of the inner layer may be greater or lesser, for instance, between two hundredths of an inch (0.02 inches) and three hundredths of an inch (0.03 inches), between two hundredths of an inch (0.02 inches) and twenty-five thousandths of an inch (0.025 inches), between twenty-two thousandths of an inch (0.022 inches) and twenty-four thousandths of an inch (0.024 inches), greater than two hundredths of an inch (0.02 inches), or less than three hundredths of an inch (0.25 inches). In some examples, the thickness of the inner layer 414 (i.e., the distance between the inner diameter and the outer diameter of the inner layer) is between five thousandths of an inch (0.005 inches) and two hundredths of an inch (0.02 inches), between one hundredth of an inch (0.01 inches) and two hundredths of an inch (0.02 inches), between five thousandths of an inch (0.005 inches) and fifteen thousandths of an inch (0.015 inches), between one hundredth of an inch (0.01 inches) and fifteen thousandths of an inch (0.015 inches), greater than five thousandths of an inch (0.005 inches), or less than two hundredths of an inch (0.02 inches).

The outer layer 416 of the elongated tube 410 also includes an outer diameter (e.g., d1) and an inner diameter (e.g., d2), the outer diameter forming the outer diameter of the elongated tube of the catheter body. As mentioned previously, in some examples the diameter of the outer layer 416 can be reduced by heating the outer layer over the inner layer 414 during assembly of the catheter. For instance, as described above with respect to FIG. 3, the inner diameter of the outer layer 416 (e.g., the second tube 316) could be approximately twenty-eight thousandths of an inch (0.028 inches) before the catheter is assembled (e.g., before heating of the outer layer). In some examples, the outer diameter of the outer layer 416 could be approximately five hundredths of an inch (0.05 inches) before the catheter is assembled. However, in some examples the outer diameter of the outer layer 416 may be greater or lesser, for instance, between four hundredths of an inch (0.04 inches) and six hundredths of an inch (0.06 inches), between forty-five thousandths of an inch (0.045 inches) and fifty-five thousandths of an inch (0.055 inches), greater than five hundredths of an inch (0.05 inches), or less than five hundredths of an inch (0.05 inches) before the catheter is assembled.

In some examples, the inner diameter of the outer layer 416 is approximately twenty-three thousandths of an inch (0.023 inches) after assembly of the catheter (e.g., after heating the outer layer). However, in some examples the inner diameter may be greater or lesser, for instance, between two hundredths of an inch (0.02 inches) and three hundredths of an inch (0.03 inches), between two hundredths of an inch (0.02 inches) and twenty-five thousandths of an inch (0.025 inches), greater than two hundredths of an inch (0.02 inches), or less than twenty-five thousandths of an inch (0.025 inches) after assembly of the catheter. In some examples, the outer diameter of the outer layer 416 could be approximately four hundredths of an inch (0.04 inches) after the catheter is assembled. In some examples, the outer diameter of the outer layer 416 is no greater than approximately forty-four thousandths of an inch (0.044 inches) after the catheter is assembled. However, in some examples the outer diameter of the outer layer 416 may be greater or lesser, for instance, between three hundredths of an inch (0.03 inches) and five hundredths of an inch (0.05 inches), between thirty-five thousandths of an inch (0.035 inches) and forty-five thousandths of an inch (0.045 inches), between three hundredths of an inch (0.03 inches) and four hundredths of an inch (0.04 inches), between thirty-five thousandths of an inch (0.035 inches) and four hundredths of an inch (0.04 inches), greater than four hundredths of an inch (0.04 inches), or less than four hundredths of an inch (0.04 inches) after the catheter is assembled. In some examples, heating of the outer layer 416 may cause a reduction in the outer diameter of the outer layer by between 10% and 25% compared to an outer diameter of the outer layer prior to heating (e.g., prior to assembly of the catheter). In some examples, the heating of the outer layer 416 may cause no less than 5%, no less than 10%, or no less than 15% of a reduction in the outer diameter of the outer layer compared to the outer diameter of the outer layer prior to heating. In some examples, heating of the outer layer 416 causes the outer diameter of the outer layer to be up to 25% smaller than the outer diameter of the outer layer prior to heating.

The thickness of the outer layer 416 may be relatively small, such that the outer layer secures the electrode assembly onto the inner layer 414 without substantially increasing the diameter of the catheter's distal end. In some examples, the thickness of the outer layer 416 is less than the thickness of the inner layer 414. In some examples, the thickness of the outer layer 416 is approximately equal to the thickness of the emitter band(s) 420. In some examples, the thickness of the outer layer 416 (i.e., the distance between the inner diameter and the outer diameter of the outer layer) could be between five thousandths of an inch (0.005 inches) and one hundredth of an inch (0.01 inches). In some examples, the thickness of the outer layer 416 or a portion of the outer layer is greater than five thousandths of an inch (0.005 inches) or greater than one hundredth of an inch (0.01 inches). In some examples, the thickness of the outer layer 416 or a portion of the outer layer is less than one hundredth of an inch (0.01 inches) or less than five thousandths of an inch (0.005 inches). In one or more examples, the outer layer is thinner at a location of an emitter band than locations where there is no emitter band.

In various embodiments, the discontinuous structure of emitter band allows the assembled catheter to have a smaller diameter (and thus a smaller crossing profile) than possible with catheters with continuous emitter bands. Some catheters may have a crossing profile (or distal region diameter) no greater than forty-four thousandths of an inch (0.044 inches). In some embodiments, the catheter's crossing profile (or distal region diameter) may be no greater than four hundredths of an inch (0.040 inches). In some examples, the crossing profile of the catheter is between three hundredths of an inch (0.03 inches) and four hundredths of an inch (0.04 inches).

FIG. 4A illustrates an exemplary catheter 400a having an emitter band 420 that is at least partially embedded within the outer layer 416 of the elongated tube 410. For instance, the bottom surface and lateral sides of the emitter band 420 may be surrounded by the outer layer 416, while the top surface of the emitter band 420 is not surrounded by the outer layer 416. In some examples and as described further below, the emitter band 420 may include one or more attachment features, and the attachment features are at least partially surrounded by or encapsulated within the outer layer 416. For instance, the emitter band 420 may include one or more thinned portions (e.g., thinned portions near one or more ends of the emitter band) and the thinned portions may be surrounded by or encapsulated within the outer layer 416. As seen in FIG. 4A, the wires 430, 432 may be at least partially encapsulated within the outer layer 416.

In some examples, when the catheter 400a has been assembled, the outer layer 416 of the elongated tube 410 is flush with the emitter band 420 of the electrode assembly. For instance, the outer diameter of the outer layer 416 may be approximately equal to the outer diameter of the emitter band 420. In some examples, material is removed from the outer layer 416 until the outer diameter of the emitter band 420 is flush with the outer diameter of the outer layer of the elongated tube 410. For instance, the exemplary catheter 400a shown in FIG. 4A could be formed by removing material from the outer layer 416 of the catheter 400b of FIG. 4B until the diameter of the emitter band 420 is equal to the outer diameter of the outer layer. In some examples, some portions of the outer layer 416 are removed to reveal some portions of the emitter band 420, while other portions of the emitter band remain encapsulated by the outer layer. For instance, the ends of the emitter band 420 may be fully surrounded or encapsulated by the outer layer 416, while a connecting region of the emitter band between the ends is not encapsulated by the outer layer. Additional material may be removed from the outer layer 416 to form a recess 418 (e.g., a hole) at a spark gap between the emitter band and wire to allow shock waves emitted at the spark gap to propagate outward from the catheter body.

FIG. 4B illustrates an exemplary catheter 400b having an emitter band 420 that is at least partially encapsulated within the outer layer 416 of the elongated tube 410. For instance, at a least a portion of the emitter band 420 may be encapsulated on all sides by the outer layer 416, while another portion of the emitter band (e.g., a portion of the emitter band positioned near wire 430 and forming an electrode of an electrode pair) is not encapsulated by the outer layer 416. As specified above, in some examples the emitter band 420 includes one or more attachment features, and the attachment features are encapsulated by the outer layer 416. In some examples, the wires 430, 432 are at least partially encapsulated within the outer layer 416.

In some examples, when the catheter 400b has been assembled, the outer layer 416 overlays at least a portion of the outer surface of the emitter band 420. For instance, a thin layer of the outer layer 416 may surround the outer surface of the emitter band 420 or a portion of the outer surface of the emitter band 420. The diameter of the emitter band 420 may be less than the diameter of the outer surface of the outer layer 416. As mentioned above with respect to FIG. 4B, a portion of material could be removed at a spark gap 433 between the emitter band 420 and wire 430 to form a recess 418 that allows shock waves emitted at the spark gap to propagate outward from the catheter body. As described previously, a shock wave catheter can include one or more discontinuous emitter bands mounted around an elongated tube. FIGS. 5A-5B illustrate exemplary discontinuous emitter bands 520a-520b that can be included in a shock wave catheter, such as any of the catheters 100, 200, 300 and 400a-400b shown in FIGS. 1-4 and described supra. The emitter bands 520a-520b include an outer surface 521, an inner surface 522, and an outer edge 523 that connects the outer surface and the inner surface. An exemplary discontinuous emitter band 520a-520b can be generally crescent-shaped, having a first end 524 and a second end 525 separated by a gap 527 with a connecting region 526 extending therebetween (see, e.g., the emitter band 520a of FIG. 5A). However, in other examples, an exemplary emitter band 520a-520b may form a coil shape, for example a helical or spiral coil shape and/or a keyring shape (see, e.g., the emitter band 520b of FIG. 5B). The discontinuous form of the emitter band 520a-520b provides various advantages over continuous emitter bands, including the ability to adjust the diameter of the band during fabrication of the catheter. The discontinuous form of the emitter band 520a-520b also allows the band to flex by a small amount during use of a catheter, e.g., to give the distal end of the catheter increased radial flexibility compared to catheters that include continuous emitter bands. In some examples, the width or maximum width of the emitter band 520a-520b at the connecting region 526 between the ends is less than the width or maximum width of the emitter band at the first 424 and second 425 ends. Reducing the width of the emitter band 520a-520b may further improve the flexibility of the emitter band and the radial flexibility of an electrode assembly. However, in other examples the width of the emitter band 520a-520b is constant.

In some examples, when the emitter band 520a-520b is mounted over the elongated tube of a catheter, the band encircles only a portion of the circumference of the elongated tube and a gap 527 exists between the first end 524 and the second 525 end. In some examples, the emitter band 520a-520b is configured to encircle at least 180 degrees of the inner layer of an elongated tube of a catheter. However, the emitter band 520a-520b may be configured to encircle more or less of the inner layer. For instance, the emitter band 520a-520b could be configured to encircle at least 120 degrees of the elongated tube, at least 150 degrees of the elongated tube, at least 210 degrees of the elongated tube, at least 240 degrees of the elongated tube, at least 270 degrees of the elongated tube, at least 300 degrees of the elongated tube, or at least 330 degrees of the elongated tube. In some examples, the gap 527 between the first end 514 and second 525 end of the emitter band 520a-520b is large enough that current does not flow between the first and the second end of the band when voltage pulses are applied to the electrode assembly.

In other examples, a discontinuous emitter banda-520b is configured to encircle the entire circumference of the inner layer one or more times. For instance, the discontinuous emitter band 520a-520b could be configured to encircle greater than 360 degrees around the circumference of the elongated tube, greater than 450 degrees, greater than 540 degrees, greater than 630, or greater than 720 degrees around the circumference of the elongated tube. In some examples, the emitter band 520a-520b winds around the elongated tube one or more times, for instance, two times, three times, four times, or more than four times. In such examples, the emitter band 520a-520b could be spiral-shaped and/or keyring-shaped.

As described above, the emitter band 520a-520b can be expanded to assume a relatively larger-diameter shape and compressed to assume a relatively smaller-diameter shape. For instance, the diameter of the band 520a-520b may be expanded by applying an outward force on the inner surface 522 of the band to increase the diameter of the band. In some examples, the diameter of the emitter band 520a-520b may be increased before mounting the band over a first tube during assembly of a catheter. Similarly, the diameter of the band 520a-520b may be compressed by applying an inward force on the outer surface 521 of the band to decrease the diameter of the band. For instance, the diameter of the band 520a-520b can be decreased to fit more tightly around the first tube assembly of a catheter and prior to application of the second tube. In some examples, the heating and shrinking of the second tube over the first tube to form the inner and outer layers of the elongated tube causes the diameter of the emitter band 520a-520b to decrease.

In some examples, the diameter of the band 520a520b may be increased or otherwise adjusted by hand, e.g., to increase the diameter of the band so it is slidable over the first tube or to decrease the diameter of the band once it has been mounted over the first tube. Accordingly, the force required to change the diameter of the band 520a-520b may be less than or equal to the force that can be applied by the average human hand. In another example, the force required to change the diameter of the band may be less than or equal to the force applied by the second tube when the second tube is shrunk to fit over the first tube during fabrication of a catheter. Advantageously, the emitter band 520a-520b may be formed from a relatively thin material that allows the diameter of the band to be easily manipulated during fabrication of the catheter.

At least a portion of the emitter band 520a-520b is formed from a conductive material, such as a metal. In more specific examples, the emitter band 520a-520b is formed from stainless steel, copper, tungsten, platinum, palladium, molybdenum, and/or an alloy or alloys thereof. At least a portion of the emitter band forms a first electrode of an electrode pair. For instance, in some examples, the outer edge 523 of the emitter band 520a-520b is a first electrode of an electrode pair, and a second electrode is formed from a conductive end of a wire placed proximate to the outer edge.

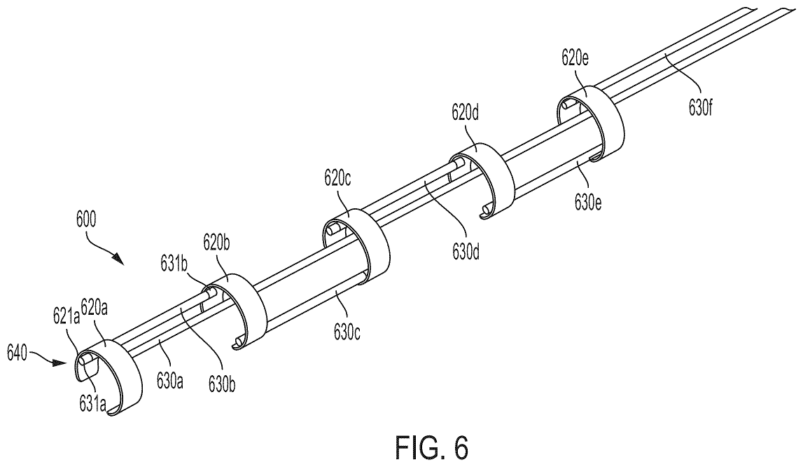

As described above, an emitter band can be included in an electrode assembly of a shock wave catheter. FIG. 6 illustrates an exemplary shock wave electrode assembly 600 constructed using a plurality of the crescent-shaped emitter bands 520a shown in exemplary FIG. 5A. As seen in FIG. 6, an electrode assembly could include a plurality of emitter bands electrically connected to each other by a plurality of conductive wires. For instance, the electrode assembly pictured in FIG. 6 includes a first emitter band 620a, a second emitter band 620b, a third emitter band 620c, a fourth emitter band 620d, and a fifth emitter band 620e connected by a respective first wire 630a, second wire 630b, third wire 630c, fourth wire 630d, fifth wire 630e, and sixth wire 630f. An outer edge of each emitter band 620a-620e (e.g., outer edge 621a of emitter band 620a) can form a first electrode in an electrode pair, and an end of one or more of the wires 630a-630f can be positioned near the outer edge of the emitter bands (i.e., spaced from the outer edge by a spark gap) to form a second electrode of an electrode pair. In some examples, the proximal ends of one or more of the wires 630a-630f (e.g., the first wire 630a and the sixth wire 630f) are connected to a pulsed voltage source, such as the voltage source 30 pictured in FIG. 1. In a particular example, the first wire 630a is connected to a negative terminal of the voltage source, and the sixth wire 630f is connected to a positive terminal of the voltage source or to ground.

In some examples, the conductive wires 630a-630f of the electrode assembly are copper wires, optionally insulated by an insulating layer (e.g., an insulating layer formed from a compliant polymer. However, in other examples the conductive portions of the wires 630a-630f are formed from another conductive material, such as a different metal. In some examples, at least a portion of the conductive portions of the wires 630a-630f (e.g., the ends of the conductive portions that are positioned near the emitter bands and form electrodes of an electrode pair) are formed from a different material than the remaining conductive portions of the wire. For instance, at least a portion of the conductive portions of the wires 630a-630f could be formed from a material that improves the resistivity and endurance of the electrode pairs. In a particular example, at least a portion of the conductive portions of the wires 630a-630f could be formed from molybdenum.

In some examples, one or more of the ends of the wires 630a-630f are positioned apart from the outer edge of the emitter bands 620a-620e by a spark gap (e.g., spark gap 640), and shock waves are generated when current flows through the electrode assembly and across the spark gaps. An emitter band and/or wire may thereby form a first and second electrode of an electrode pair, or may form electrodes of a plurality of electrode pairs. Additionally, or alternatively, one or more of the wires 630a-630f may be soldered to the emitter bands 620a-620e to provide a direct electrical connection between the wire and the emitter band. In such examples, a shock wave will not be produced when current flows between the emitter band and a wire. Accordingly, the electrode assembly may generate shock waves at some of the junctures between wires 630a-630f and emitter bands 620a-620e (i.e., at the spark gaps) and not form shock waves at other junctures (i.e., at direct electrical connections between the wires and emitter bands).

When a voltage is applied across the first 630a and sixth 630f wires, current is configured to flow through the electrode assembly to generate shock waves at one or more sparks gaps between the emitter bands 620a-620e and the conductive ends of the wires 630a-630f. For instance, in the exemplary assembly 600 shown in FIG. 6, when a voltage pulse is applied across the first 630a and sixth 630f wires, a current flows from the voltage source through the first wire 630a, then from the first wire to the first emitter band 620a. The current then flows across the first emitter band 620a and from an outer edge 621a of the first emitter band to a distal end 631a of the second wire 630b, through the second wire, and from the proximal end 631b of the second wire to the second emitter band 620b. The current then flows across the second emitter band 620b and from the outer edge of the second emitter band to a distal end of the third wire 630c, through the third wire, and from the proximal end of the third wire to the third emitter band 620c. The current may then continue to flow through the rest of the electrode assembly, i.e., from the third emitter band 620c to the fourth wire 630d, from the fourth wire to the fourth emitter band 620d, from the fourth emitter band to the fifth wire 630e, from the fifth wire to the fifth emitter band 620e, and from the fifth emitter band to the sixth wire 630f following a similar scheme. The current may then flow through the sixth wire 630f to reach ground or the positive terminal of a voltage source.

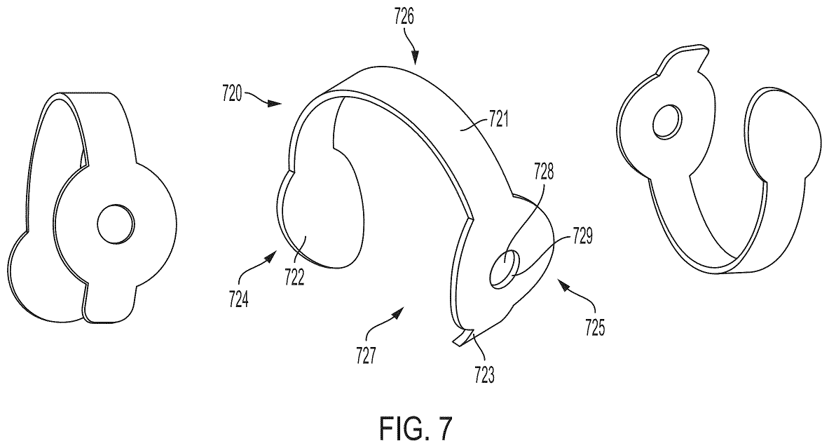

FIG. 7 illustrates another exemplary discontinuous emitter band 720 that can be included in a shock wave catheter, such as any of the catheters 100, 200, 300 and/or 400a-400b shown in FIGS. 1-4 and described supra. The emitter band 720 includes an outer surface 721, an inner surface 722, and an outer edge that connects the outer surface and the inner surface. The emitter band 720 shown in FIG. 7 may be generally similar to the emitter band 520a shown in FIG. 5A and described above. However, the exemplary discontinuous emitter band 720 is headphone-shaped, including a first enlarged region 724 at its first end, a second enlarged region 725 at its second end, and a connecting region 726 extending therebetween. In some examples, the width or maximum width of the first enlarged region 724 and the second enlarged region 725 is greater than the width or maximum width of the connecting region 726. The width of the enlarged regions 724, 725 may be selected such that repeated generation of shock waves at the emitter band 720 does not wear away the enlarged regions through the life cycle of a catheter. In some examples, an additional attachment portion 723 extends further than the first 724 and/or second 725 enlarged regions to allow the emitter band 720 to encircle a greater portion of the circumference of the elongated tube and improve the securement of the emitter band within a catheter.

At least a portion of the emitter band 720 forms a first electrode of an electrode pair. For instance, in some examples, one or more of the first enlarged region 724 and the second enlarged region 725 includes an aperture 728. Aperture 728 may have various shapes, including, for example, a circular shape, an elliptical shape, a slot shape, or a triangular shape. An inner edge 729 of the aperture 728 may form a first electrode of an electrode pair, and a second electrode of the pair is formed from a conductive end of a wire placed proximate to the inner edge. Advantageously, forming an electrode out of an inner edge 729 of the aperture 728 may cause shock waves to be directed outward through the aperture such that the shock waves propagate in an outward direction toward the enclosure of the catheter and into the body lumen where it is absorbed by the lesion. Such an emitter band configuration may emit shock waves in a manner that is less non-specific than the outer edge-firing emitter bands 520a-520b shown in FIGS. 5A-5B.

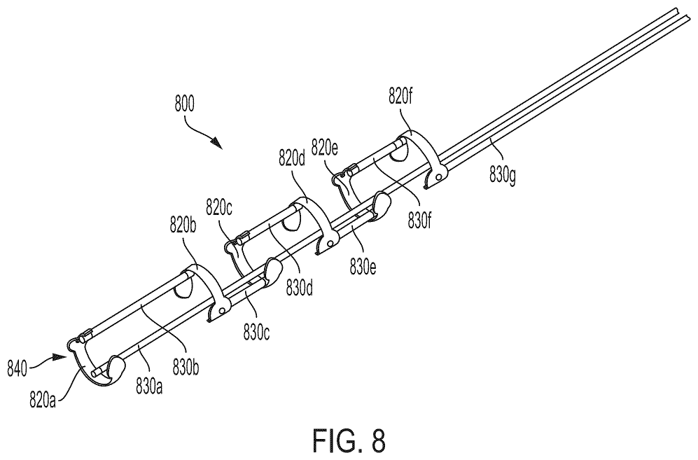

FIG. 8 illustrates an exemplary shock wave electrode assembly constructed using a plurality of the headphone-shaped emitter band 720 shown in exemplary FIG. 7. The electrode assembly 800 may be generally similar to the electrode assembly shown in FIG. 6, except that the crescent-shaped emitter bands are replaced with the headphone-shaped emitter bands of FIG. 7 and an additional emitter band has been added. As seen in FIG. 8, the electrode assembly 800 includes a first emitter band 820a, a second emitter band 820b, a third emitter band 820c, a fourth emitter band 820d, a fifth emitter band 820e, and a sixth emitter band 820f connected by a respective first wire 830a, second wire 830b, third wire 830c, fourth wire 830d, fifth wire 830e, sixth wire 830f, and seventh wire 830g. An aperture of each of the emitter bands 820a-820f can form a first electrode in an electrode pair, and a conductive end of one or more of the wires 830a-830g can be positioned near the inner edge of the aperture (i.e., spaced from the inner edge of the aperture by a spark gap) to form a second electrode of an electrode pair. In some examples, the proximal ends of one or more of the wires 830a-830g (e.g., the first wire 830a and the seventh wire 830g) are connected to a pulsed voltage source, such as the voltage source 30 pictured in FIG. 1. In a particular example, the first wire 830a is connected to a negative terminal of the voltage source, and the seventh wire 830g is connected to a positive terminal or the voltage source or to ground.

As described in relation to FIG. 6, in some examples the ends of one or more of the wires 830a-830g are positioned apart from the inner edge of the apertures of the emitter bands 820a-820f by a spark gap (e.g., spark gap 840), and shock waves are generated when current flows through the electrode assembly and across the spark gaps. Additionally, or alternatively, one or more of the wires 830a-830g may be soldered to the emitter bands 820a-820f to provide a direct electrical connection between the wire and the emitter band. In such examples, a shock wave will not be produced when current flows between the emitter band and a wire. For instance, in the electrode assembly of FIG. 8, each of the emitter bands 820a-820f may include a first enlarged region that includes an aperture, and a second enlarged region that does not include an aperture. The wires 830a-830g and emitter bands 820a-820f may be positioned in the electrode assembly such that a spark gap is formed between the ends of the wires and the aperture at the first enlarged regions, and other ends of the wires are soldered directly to the second enlarged region. Accordingly, the electrode assembly may generate shock waves at the junctures between the wires 830a-830f and the first enlarged regions of the emitter bands 820a-820g (i.e., at the spark gaps) and not form shock waves at the junctures between the wires and the second enlarged regions (i.e., at direct electrical connections between the wires and emitter bands).

When a voltage is applied across the first 830a and seventh 830g wires, current is configured to flow through the electrode assembly to generate shock waves at the sparks gaps between the emitter bands 820a-820f and conductive ends of the wires 830a-830g. For instance, in the exemplary assembly shown in FIG. 8, when a voltage pulse is applied across the first 830a and seventh 830g wires, a current flows from the voltage source through the first wire 830a, then from the distal end of the first wire to the first emitter band 820a. The current then flows across the first emitter band 820a and from the inner edge of the aperture in the first enlarged region of the first emitter band to a distal end of the second wire 830b, through the second wire, and from the proximal end of the second wire to the second enlarged region of the second emitter band 820b. The current then flows across the second emitter band 820b and from the inner edge of the first enlarged region of the second emitter band to a distal end of the third wire 830c, through the third wire, and from the proximal end of the third wire to the second enlarged region of the third emitter band 820c. The current may then continue to flow through the rest of the electrode assembly, i.e., from the third emitter band 820c to the fourth wire 830d, from the fourth wire to the fourth emitter band 820d, from the fourth emitter band to the fifth wire 830e, from the fifth wire to the fifth emitter band 820e, from the fifth emitter band to the sixth wire 630f, from the sixth wire to the sixth emitter band 620f, and from the sixth emitter band to the seventh wire 830g following a similar scheme. The current may then flow through the seventh wire 830g to reach ground or the positive terminal of a voltage source.

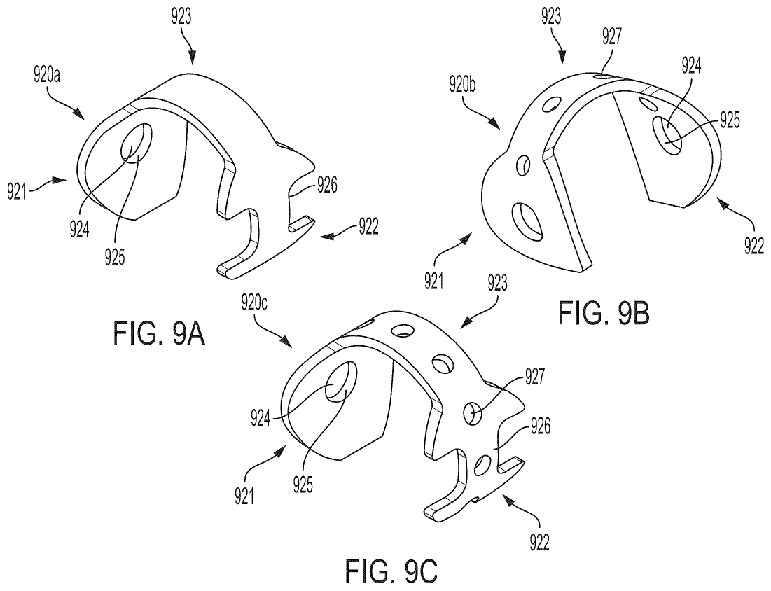

FIGS. 5A-5B and FIG. 7 illustrate three exemplary discontinuous emitter bands that could be included in an electrode assembly of a shock wave catheter, however an emitter band could be shaped differently or include additional or alternative features as the bands pictured in FIGS. 5A-5B and FIG. 7. For instance, FIGS. 9A-9C illustrate various additional examples of discontinuous emitter bands 920a-920c that could be incorporated in a shock wave catheter, such as any of the shock wave catheters 100, 200, 300, 400a, and/or 400b shown in FIGS. 1-4B. The emitter bands 920a-920c may be generally similar to the emitter bands shown in FIGS. 5A-5B and FIG. 7, having a first end (e.g., first enlarged region 921), a second end (e.g., second enlarged region 922), and a connecting region 923 extending therebetween. In some examples, a width or maximum width of the first enlarged region 921 and/or the second enlarged region 922 is greater than a width or maximum width of the connecting region 923. The bands 920a-920c in FIGS. 9A-9C further include features that facilitate fabrication of a catheter and the use of the catheter to treat lesions in body lumens.

For instance, as seen in FIG. 9A, an emitter band 920a may include one or more slots 926 in the band for attaching (e.g., soldering) one or more wires to the emitter band. More specifically, in some examples, the emitter band 920a could include a first enlarged region 921 at a first end, a second enlarged region 922 at a second end, and a connecting region 923 therebetween. The first enlarged region 921 and the second enlarged region 922 are widened compared to the connecting region 923. In some examples, the first enlarged region 921 includes an aperture 924 and an inner edge 925 of the aperture forms a first electrode of an electrode pair. In some examples, the second enlarged region 922 includes one or more slots 926 or gaps extending into the lateral edges of the second enlarged region 922 for attaching one or more wires to the emitter band. In some examples, first enlarged region 921 and/or second enlarged region 922 include one or more slots such as slots 926 or gaps extending into the lateral edges of the respective enlarged region for attaching one or more wires to the emitter band. The one or more slots 926 may be configured such that a wire can be electrically connected (e.g., soldered directly) to the emitter band 920a at the second enlarged region 922 proximate to the slot(s).

FIG. 9B illustrates another exemplary emitter band 920b including a plurality of securement holes 927. The emitter band 920b may include a first enlarged region 921 at a first end, and/or second enlarged region 922 at a second end and a connecting region 923 extending therebetween. The first enlarged region 921 and/or the second enlarged region 922 may be widened compared to the connecting region 923, and each of the enlarged regions may include a respective aperture (e.g., aperture 924). First enlarged region 921 and/or second enlarged region 922 may include an aperture (e.g., aperture 924) that forms an elliptical shape. An inner edge (e.g., inner edge 925) of each of the apertures may form an electrode of an electrode pair. Accordingly, the emitter band 920b shown in FIG. 9B may be used as an electrode in a plurality of electrode pairs and may generate shock waves at spark gaps proximate to each of the enlarged regions 921 and/or 922. In alternative embodiments, an emitter band may include more than two apertures whose inner edges form an electrode of an electrode pair.

The emitter band 920b pictured in FIG. 9B may further include a plurality of securement holes 927 along the connecting region of the band. The securement holes 927 may facilitate the embedding or encapsulation of the band 920c between the layers of the elongated tube during fabrication of a catheter. For instance, in some examples, at least a portion of the inner layer or outer layer of the elongated tube is embedded within the securement holes 927 when the outer layer is sealed to the inner layer. In some examples, an emitter band, such as the emitter band 920b, may include additional or alternative means of facilitating securement of the emitter band to the layers of catheter. For instance, in some examples, an emitter band could include raised features, such as protrusions, ridges and/or studs, configured to extend outwardly from the band and into one or more of the polymer layers to facilitate securement of the band within the polymer layers. In some other examples, the emitter bands could be modified (e.g., by machining the surface of the emitter band) to include inward depressions, dimples, ridges, and/or other features that improve the adherence of the band within the polymer layer.

FIG. 9C illustrates yet another exemplary emitter band 920c that includes aspects of each of the emitter bands 920a and/or 920b shown in FIGS. 9A and 9B. For instance, the emitter band 930c includes a first end including a first enlarged region 921 having an aperture 924, similar to the emitter band 920a shown in FIG. 9A. An inner edge 925 of the aperture may form a first electrode of an electrode pair. The emitter band further includes a second end including a second enlarged region 922, the second enlarged region including slots 926 extending into the lateral edges of the second enlarged region, similar to the band shown in FIG. 9A. A connecting region 923 extends between the first enlarged region and the second enlarged region, and a plurality of securement holes 927 are positioned along the connecting region. In some examples, and as seen in FIG. 9C, one or more of the securement holes 927 are positioned in the first or second enlarged regions 921, 922.

While FIGS. 5A-5B, FIG. 7 and FIGS. 9A-9C illustrate five exemplary emitter bands that could be included in a shock wave electrode assembly of a catheter, an emitter band of the present disclosure may incorporate aspects of any of the emitter bands described here. Accordingly, the features and characteristics of the emitter bands may be combined, interchanged, and/or removed without departing from the present disclosure. Further, an electrode assembly could include a plurality of different emitter bands, each of the emitter bands including various features and characteristics of the emitter bands described here.

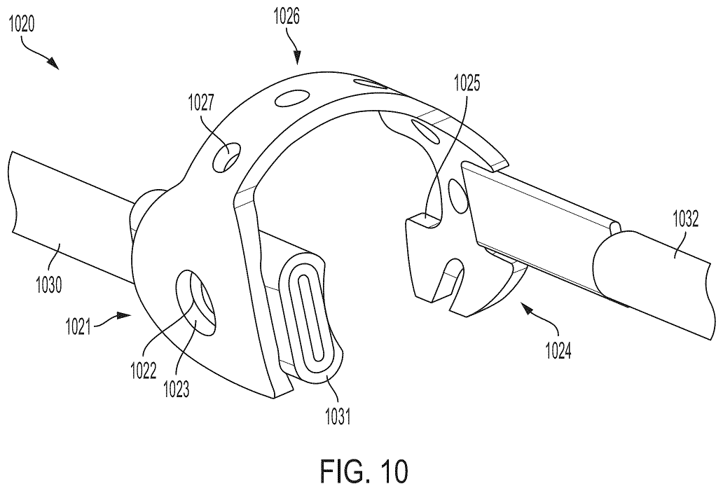

As described previously, the emitter bands may be arranged with various wires and other components to form an electrode assembly of a shock wave catheter. FIG. 10 illustrates an exemplary emitter band 1020 that is electrically connected (e.g., by soldering or with a conductive adhesive) to a first wire 1030 and a second wire 1032. The emitter band 1020 includes features similar to the emitter band 920c pictured in FIG. 9C. For instance, the first end of the emitter band includes a first enlarged region 1021 that includes an aperture 1022, with an inner edge 1023 of the aperture forming a first electrode of an electrode pair. The first wire 1030 is soldered to the first enlarged region 1021 of the emitter band 1020, with a distal end of the first wire forming a second electrode of the electrode pair. An aperture in the distal end of the first wire 1030 is positioned proximate to the enlarged region 1021 and separated from the inner edge 1023 of the aperture 1022 by a spark gap, such that when a current flows from the first wire 1030 to the first enlarged region 1021, a shock wave is generated across the spark gap. The second end of the emitter band includes a second enlarged region 1024 that includes slots 1025 for soldering a wire to the emitter band, e.g., the second wire 1032 soldered to the second enlarged region 1024 of the band 1020 pictured in FIG. 10. The second wire 1032 is soldered to the second enlarged region 1024 of the emitter band 1020 at a slot 1025 to provide a direct electrical connection between the emitter band 1020 and the second wire 1032. The emitter band 1020 also includes a connecting region 1026 between the first 1021 and second 1024 enlarged region. One or more securement holes 1027 are positioned on the connecting region 1026 and the second enlarged region 1024 to facilitate securement of the emitter band 1020 between the inner and outer layers of the catheter body.

In some examples, the soldering of a wire to an emitter band provides a direct electrical connection between the conductive wire and the band (i.e., the conductive portion of the wire may be soldered directly to the emitter band). When the conductive portion of the wire is soldered directly to the emitter band, current can flow directly from the wire to the band without traversing a spark gap and forming a shock wave. For instance, as shown in FIG. 10, the conductive portion of the second wire 1032 is soldered directly to the second enlarged region 1024 of the emitter band 1020.

In some examples, the soldering of a conductive wire (e.g., either of wires 1030 and/or 1032 to the emitter band 1020 does not provide a direct electrical connection between the band and the wire (i.e., such that current does now flow between the wire and the emitter band at the soldered portion and instead flows across a spark gap). For instance, the soldered portion of a wire could be electrically separated from a conductive portion of the wire along which current flows when a voltage is applied to the wire. Accordingly, when the wire is soldered to the emitter band, the conductive portion of the wire may be spaced apart from the emitter band at a spark gap, such that the conductive portion of the soldered wire and the emitter band form an electrode pair for generating shock waves at the spark gap. For instance, as shown in FIG. 10, the first wire 1030 is soldered to the first enlarged region 1021 of the emitter band 1020, and the conductive portion of the first wire is separate from the soldered portion and positioned inside the aperture 1022 and separated from the emitter band by a spark gap. In some examples, an insulating layer 1031 is provided on one or more of the wires 1030 and/or 1032 to prevent current from flowing to and/or from portions of the wire covered by the insulating layer. The insulating layer 1031 may be formed from a non-conductive material, such as an insulating polymer (e.g., polyimide). In some examples, an aperture in the insulating layer 1031 defines the spark gap between a wire (e.g., wire 1030) and an electrode formed from the emitter band.

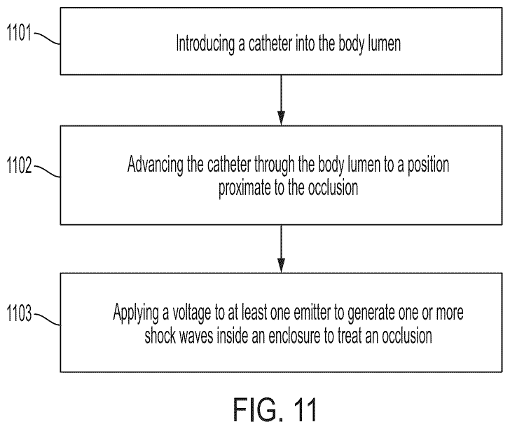

The exemplary catheters described herein may be used to treat a lesion in a body lumen during a shock wave procedure. FIG. 11 illustrates a method of treating an occlusion with a catheter, such as any of the catheters described above with respect to FIGS. 1-10. As described previously, a catheter may include an elongated tube with a guide wire lumen, one or more electrode pairs mounted along the elongated tube, and an enclosure surrounding the one or more electrode pairs.

At step 1101, the method includes introducing a catheter into a body lumen. In some examples, introducing the catheter into the body lumen includes advancing a guide wire from an entry site on a patient (e.g., an artery in the groin area of the leg) to the target region of a vessel (e.g., a region having calcified plaques that need to be broken up), and advancing the catheter into the body lumen over the guide wire.

At step 1102, the method includes advancing the catheter through the body lumen to a position proximate to the occlusion. The location of the shock wave device may be determined by x-ray imaging and/or fluoroscopy. In some examples, advancing the catheter through the body lumen includes advancing the catheter until the enclosure of the catheter or one or more of the electrode pairs within the enclosure is proximate to the occlusion. In some examples, the enclosure of the catheter is in a collapsed or folded state when the catheter is inserted and advanced through the body lumen. When the shock wave device reaches the target region, the enclosure may be inflated by a conductive fluid (e.g., saline and/or saline mixed with an image contrast agent) until the enclosure contacts the walls of the body lumen and/or the occlusion. For example, the enclosure may be inflated to a pressure of 1 atm to 5 atm. In other examples where the enclosure is not an inflatable structure, the enclosure is filled with a conductive fluid and does not substantially increase in size before beginning a shock wave treatment.

At step 1103, the method includes applying voltage pulses across one or more of the electrode pairs to generate one or more shock waves inside the enclosure to treat the occlusion. Repeated shock waves may be generated to break up calcified plaques of the occlusion. The progress of the plaque break-up may be monitored by x-ray and/or fluoroscopy. If the occlusion is longer than the length of the catheter that includes the electrode pairs, and/or if the occlusion is too far away from the electrode pairs to receive the full force of the generated shock waves, the catheter may be moved further along the length of the vessel and additional shock waves can be delivered. For example, the catheter may be stepped along the length of a blood vessel and shock waves can be delivered sequentially at various locations along the vessel to break up more complex occlusions and larger regions of calcified plaques. The electrode pairs of the catheter may be connected in series and configured to fire together, such that a single voltage pulse causes shock wave generation at each of the electrode pairs of the catheter. In other examples, one or more of the electrodes may be connected to separate high voltage channels, allowing an operator of the catheter to activate a subset of the electrodes simultaneously and/or sequentially. In such examples, an operator may generate shock waves first at a distal subset of the electrode pairs, and subsequently at a proximal subset of the electrode pairs, or in whatever desired order to treat the occlusion. Once the occlusion has been sufficiently treated, the enclosure may be inflated further or deflated, and the catheter and guide wire may be withdrawn from the patient.

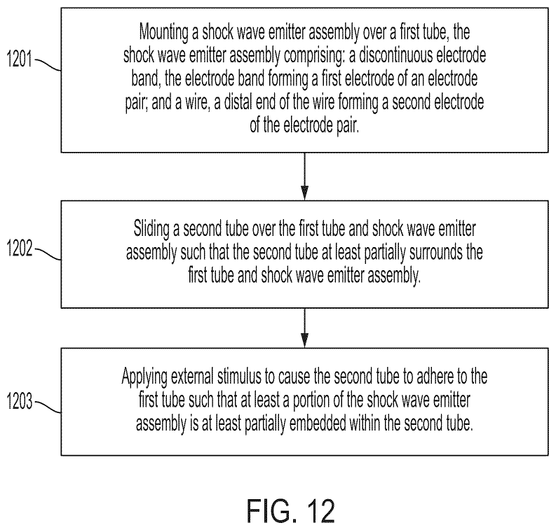

FIG. 12 illustrates an exemplary method of fabricating a catheter, such as any of the catheters described above with respect to FIGS. 1-10. As described previously, the catheter may include a shock wave electrode assembly, a first tube, a second tube, and an enclosure. The shock wave electrode assembly may include a plurality of emitter bands and a plurality of wires arranged to form various electrode pairs in the assembled catheter. More specifically, in some examples the emitter bands include discontinuous or semi-circular emitter bands, and a portion of each of the emitter bands forms a first electrode of an electrode pair. In some examples, the ends of the wires, which may be insulated copper wires, form second electrodes of the electrode pairs. The first tube and the second tube may be extruded polymer tubes, such as extruded tubes formed of PTFE, Pebax, or some other compliant polymeric material.

At step 1201, the method includes mounting a shock wave electrode assembly over a first tube of the catheter. In some examples, mounting the shock wave electrode assembly over the first tube includes extending the wires longitudinally along the first tube and abutting the first tube, and sliding the emitter bands to surround the wires and the first tube. In some examples, the method further includes electrically connecting (e.g., by soldering or with a conductive adhesive) one or more of the wires to a portion of one or more of the emitter bands. In some examples, the first tube includes attachment features, such as grooves for retaining the wires or indentation for embedding the emitter bands, and mounting the electrode assembly over the first tube includes, e.g., inserting the wires into the grooves of the first tube and/or positioning the emitter bands over the indentations. In such examples, at least a portion of the inner layer may be removed prior to mounting the electrode assembly on the inner layer (e.g., by ablating the inner layer to create the grooves, indentations and/or other attachment features). In some examples, the surface of the first tube is etched to improve the adhesion of the electrode assembly and/or the second tube to the first tube.

The emitter bands may be positioned at any desired locations along the distal end of the catheter. For instance, in some examples the emitter bands are mounted at evenly spaced locations along the distal end of the first tube. However, in other examples the emitter bands may be mounted in various groupings along the first tube, e.g., mounted in a distal grouping, a central grouping, and/or a proximal grouping. In some examples, mounting the shock wave electrode assembly includes compressing the emitter bands to secure the emitter bands over the wires and the first tube. For instance, the emitter bands may be provided in a relatively larger-diameter state, and mounting the electrode assembly could include sliding the emitter bands over the first tube and then compressing the emitter bands into a relatively smaller-diameter state. In some examples, the method includes compressing the emitter bands by hand.

At step 1202, the method includes sliding a second tube over the first tube and shock wave electrode assembly such that the second tube at least partially surrounds the first tube and shock wave electrode assembly. However, in other examples, step 1202 could include applying a sheet of a polymeric material around the first tube and the shock wave electrode assembly or applying a liquid polymeric material over the first tube and the shock wave electrode assembly. In such examples, the polymeric material may be processed (e.g., cured with heat, with UV light, or with some other stimulus) to seal the polymeric material over the first tube.

At step 1203, the method includes applying heat to cause the second tube to adhere to the first tube such that at least a portion of the shock wave electrode assembly is at least partially encapsulated within the second tube. In some examples, applying an external stimulus (e.g., heat, light, magnetic field, and/or solution) causes the second tube to shrink over the first tube to at least partially surround or encapsulate the wires and emitter bands of the electrode assembly. In these examples, the inner diameter of the second tube decreases. In other examples, both the inner diameter and the outer diameter of the second tube decrease upon application of external stimulus. The second tube may be made of a shape-memory polymer. In some examples, adhering the second tube to the first tube causes the diameter of the emitter bands to decrease. In other examples, the method further comprises pressing the second tube and/or electrode assembly inward toward the first tube.

In some examples, at least a portion of the material of the first or second tube can be removed during fabrication of the catheter. For instance, regions of material near the electrode pairs (e.g., material within the spark gap between the electrodes of an electrode pair) can be removed to allow conductive fluid to flow to the gap and shock waves that form between the electrodes to propagate outwardly from the catheter body. In such examples, the method may further include removing a portion of the material of the second tube to reveal spark gaps between the emitter bands and the wires. Additionally, or alternatively, material can be removed from the first and/or second tube to shape the catheter body. For instance, the material of the second tube may be removed to reveal portions of the emitter bands or to make the surface of the second tube flush with the outer surface of the emitter bands. In some examples, the method includes removing material from the first and/or second tube to shape the distal tip of the catheter into a point.

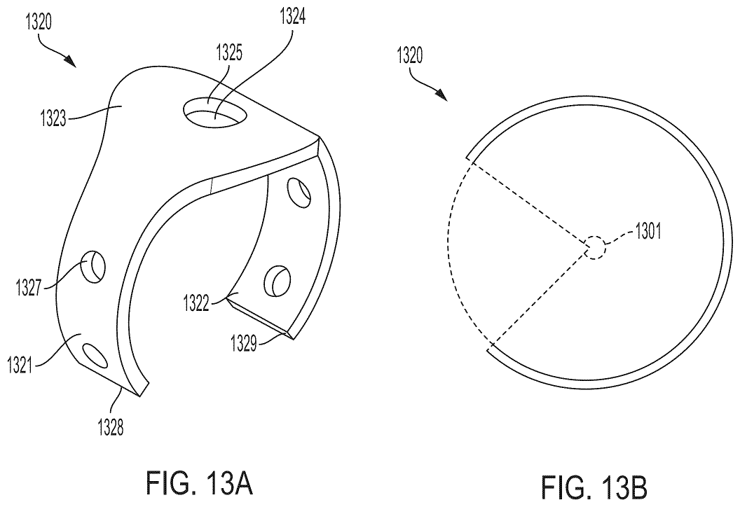

FIGS. 13A-13B illustrate another exemplary emitter band 1320 that can be incorporated into a shock wave catheter, such as any of the shock wave catheters 100, 200, 300, 400a, and/or 400b shown in FIGS. 1-4B. In this example, the discontinuous emitter band 1320 includes an enlarged central region 1323 having an aperture 1324 whose inner edge 1325 forms an electrode of an electrode pair (similar to electrodes formed by the apertures in the emitter bands described above with respect to FIGS. 7, and 9A-9C). Aperture 1324 may have a circular shape as shown, or may have another shape, such as an elliptical shape. In some examples, the enlarged region 1323 is in a central portion of the emitter band 1320. The discontinuous emitter band 1320 may include one or more leg regions. For example, two opposing leg regions (i.e., the first leg region 1321 and the second leg region 1322) that may each be narrower than the enlarged central region 1323 may extend from the enlarged region and form opposite ends of the emitter band. Each of the leg regions 1321 and/or 1322 may include a respective leg end 1328. In some examples, the first leg end 1328 of the first leg region 1321 and the second leg end 1329 of the second leg region 1322 do not connect to each other. However, in other examples, the leg ends 1328 and 1329 overlap each other, such as in the coil-shaped exemplary emitter band 520b shown in FIG. 5B. In some embodiments, the leg regions 1321 and/or 1322 have a width no greater than three tenths of a millimeter (0.3 mm). In some examples, the width or maximum width of enlarged central region 1323 is greater than the width or maximum width of leg region 1321 and/or 1322. The leg regions 1321 and/or 1322 each include one or more securement structures 1327 that, similar to the securement holes described above with respect to FIGS. 9A-9C, may provide structural features to help secure the emitter band to the catheter body. As shown in FIG. 13A, in some examples the securement structures 1327 are apertures formed in the emitter band 1320, optionally in the enlarged central region 1323.

In the side view of the emitter band 1320 shown in FIG. 13B, the discontinuous emitter band defines an arc having a central angle 1301 no less than 180 degrees. In some embodiments, the central angle 1301 is no less than 270 degrees. Upon compression of the emitter band to a final diameter (e.g., a final diameter of the emitter band when the catheter is in an assembled state), the central angle 1301 increases from the uncompressed central angle. In one example, as shown in FIG. 13A, the leg regions 1321 and/or 1322 are substantially the same lengths. In other examples, one leg region is substantially longer than the other. In such examples, the enlarged region 1323 may be offset from the center of the emitter band 1320. Advantageously, these embodiments provide added durability and capability to stay secured to the catheter body even with pressure changes associated with generation of shock waves at the emitter band 1320.

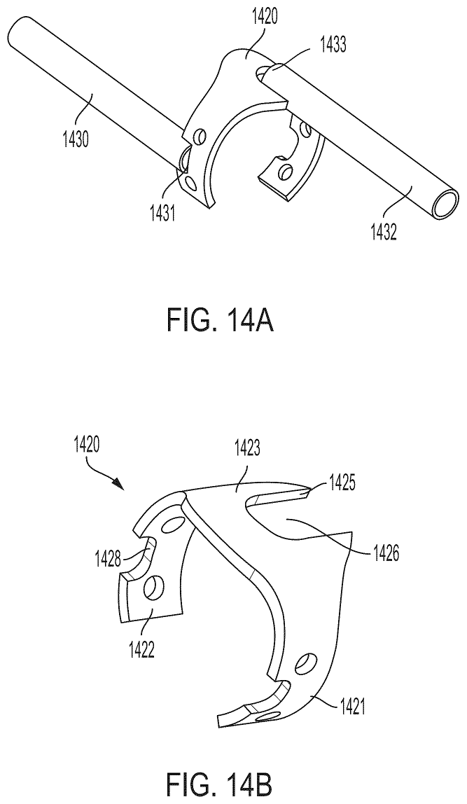

FIGS. 14A-14B illustrate another exemplary emitter band 1420 that can be incorporated into a shock wave catheter, such as any of the shock wave catheters 100, 200, 300, 400a, and/or 400b shown in FIGS. 1-4B. FIG. 14A illustrates the emitter band 1420 incorporated into an electrode assembly. The emitter band 1420 includes an enlarged region 1423 and opposing leg regions (e.g., a first leg region 1421 and/or a second leg region 1422) that extend from the enlarged region. The enlarged region 1423 includes a notch 1426 with a conductive edge 1425. For example, notch 1426 may include one or more slots for attaching one or more wires to emitter band 1420. FIG. 14A illustrates the emitter band 1420 with a first wire 1430 that is electrically connected to the emitter band and a second wire 1432 that has a conductive end 1433 that is spaced apart, e.g., by at least one millimeter (1 mm), from the conductive edge 1425 of the enlarged region by a spark gap. In one or more embodiments, the conductive end 1433 of the second wire 1432 and the conductive edge 1425 of the notch 1426 in the enlarged region 1423 are substantially coplanar. The first wire 1430 is configured to carry electrical current to or away from the emitter band 1420; the first wire 1430 may have a conductive region 1431 that is electrically connected to the emitter band by soldering, welding, conductive adhesive, or by mechanical means. The conductive edge 1425 of the notch 1426 in the enlarged region 1423 and the conductive end 1433 of the second wire 1432 form electrodes of an electrode pair and the space between the electrodes defines a spark gap. When a sufficiently high voltage is applied across the wires 1430 and 1432, current flows across the spark gap and a shock wave is generated. In one or more embodiments, the first 1430 and/or second 1432 wires each include an insulative coating (e.g., a polyimide coating) that surrounds a conductive core. The proximal end of the first wire 1430 (i.e., the conductive end 1433) does not have the insulative coating to allow for an electrical connection to be made to the emitter band 1420. In some examples, the emitter band 1420 includes one or more slots 1428 disposed in leg region 1421 and/or 1422 of the emitter band, and in some examples the first wire 1430 is electrically connected to the emitter band 1420 at or near the slot(s) 1428. For example, one or more slots 1428 may enable attachment of one or more wires to emitter band 1420. As shown in FIGS. 14A and 14B, the emitter band includes, in one or more embodiments, securement structures 1427 (e.g., holes, ridges, grooves, and/or bumps) that facilitate the securing of the emitter band 1420 to the catheter body or layers thereof.

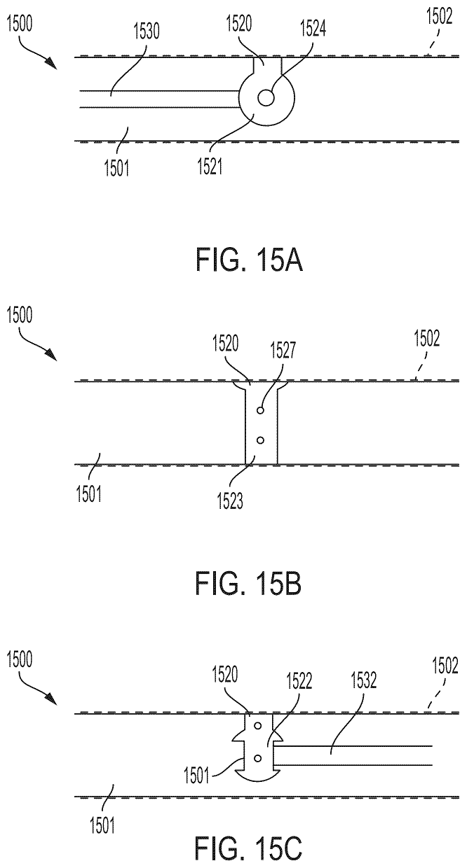

FIGS. 15A-15C illustrate an exemplary emitter assembly 1500 that can be incorporated into a shock wave catheter, such as any of the shock wave catheters 100, 200, 300, 400a, and/or 400b shown in FIGS. 1-4B. The emitter assembly 1500 includes an emitter band 1520 having a first enlarged region 1521 (shown in FIG. 15A), a connecting region 1523 (shown in FIG. 15B), and a second enlarged region 1522 (shown in FIG. 15C). The emitter band 1520 wraps at least partially around an inner layer 1501 of a catheter. An outer layer 1502 of the catheter (shown in dashed lines) at least partially surrounds and/or encapsulates the emitter band 1520 and connecting wires. The emitter band 1520 (similar to emitter band 920c or emitter band 1020 shown in respective FIGS. 9C and 10) includes an aperture 1524 with an inner edge that forms one electrode of an electrode pair. A second electrode of the electrode pair may be formed by a first conductive wire 1530. In some examples, a conductive portion of the wire 1530 is spaced apart from the inner edge of the aperture 1524 by a spark gap, where shock waves can be generated on the first enlarged region 1521. The connecting region 1522 includes securement structures 1527 (such as the securement holes shown in FIG. 15B). The second enlarged region 1522 includes a slot 1528 where a second conductive wire 1532 may be electrically connected (e.g., by soldering or with a conductive adhesive).

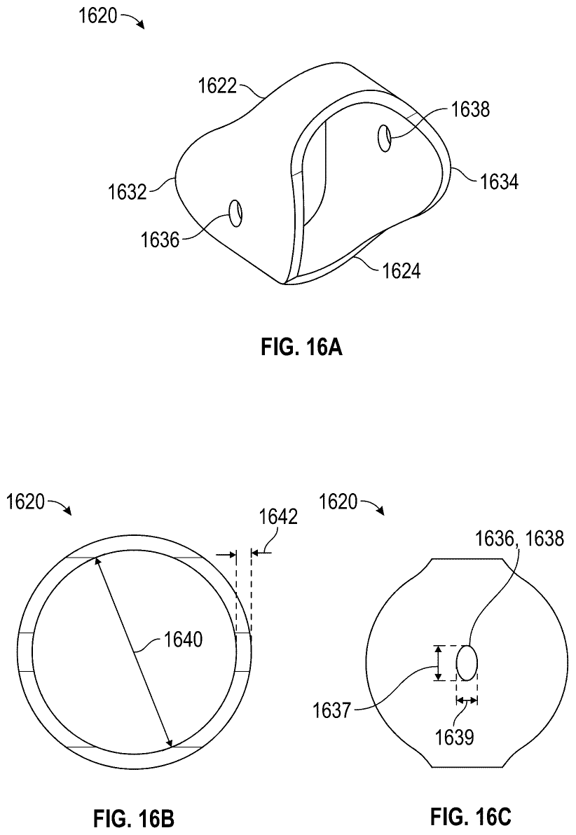

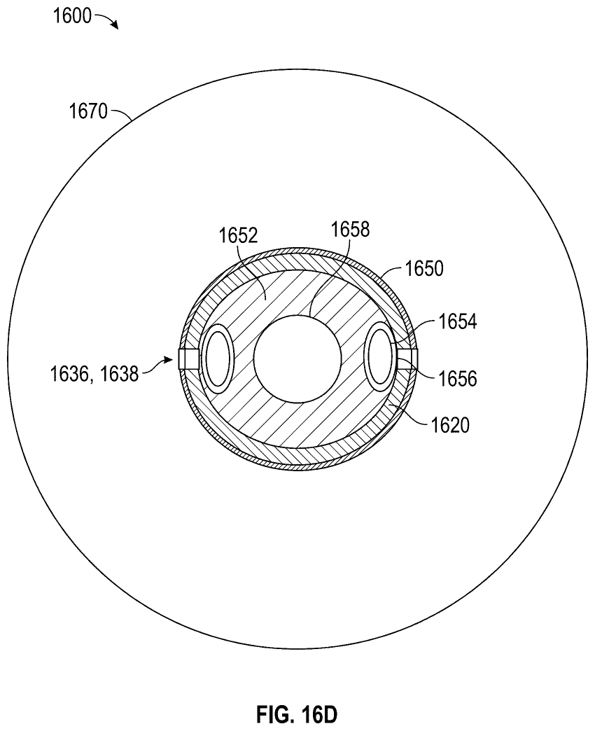

FIGS. 16A-16D illustrate another emitter band 1620 that can be incorporated into a low-profile, flexible shock wave catheter. In contrast to the emitter bands described above, the emitter band 1620 is not discontinuous (e.g. the emitter band forms a continuous, closed loop). But similarly, emitter band 1620 may be provided as part of a low-profile shock wave catheter with the emitter band being encapsulated and/or embedded within polymeric materials. As depicted in FIG. 16A, emitter band 1620 may include connecting regions 1622 and/or 1624, and/or enlarged regions 1632 and/or 1634, with connecting regions 1622 and/or 1624 extending between enlarged regions 1632 and/or 1634. Each of enlarged regions 1632 and/or 1634 may include apertures 1636 and/or 1638 respectively extending through enlarged regions 1632 and/or 1634. Including the narrow connecting regions 1622 and/or 1624 may help to make a catheter including emitter band 1620 to be more flexible and navigable. In some examples, the width or maximum width of at least one of the enlarged regions 1632 and/or 1634 may be greater than the width or maximum width of at least one of the connecting regions 1622 and/or 1624. Including enlarged regions 1632 and/or 1634 in emitter band 1620 may help to ensure that there is enough emitter material in case of erosion during shock wave generation. Enlarged regions 1632 and/or 1634 may help to ensure structural integrity of the emitter band 1620 during shock wave generation. In some examples, connecting region 1622 and/or connecting region 1624 is the same width as region 1632 and/or region 1634.

Emitter band 1620 may have an inner diameter 1640 that is at least 0.01 inches, at least 0.015 inches, at least 0.02 inches, at least 0.025 inches, at least 0.03 inches, at least 0.035 inches, at least 0.04 inches, at most 0.04 inches, at most 0.035 inches, at most 0.03 inches, at most 0.025 inches, at most 0.02 inches, at most 0.015 inches, and/or at most 0.01 inches. Emitter band 1620 may have a thickness 1642 that is at least 0.0005 inches, at least 0.001 inches, at least 0.0015 inches, at least 0.002 inches, at least 0.0025 inches, at least 0.003 inches, at least 0.0035 inches, at least 0.004 inches, at most 0.004 inches, at most 0.0035 inches, at most 0.003 inches, at most 0.0025 inches, at most 0.002 inches, at most 0.0015 inches, at most 0.001 inches, and/or at most 0.0005 inches.

As depicted in FIG. 16C, one or both of apertures 1636 and 1638 may include an elliptical shape. One or both of apertures 1636 and 1638 may have a length 1637 in the radial direction of the emitter band 1620 that is greater than a width 1639 in the axial direction of the elliptical band 1620. For example, one or both of apertures 1636 and 1638 may have a length 1637 in the radial direction of the emitter band 1620 that is at least 25%, at least 50%, at least 75%, at least 100%, at least 125%, at least 150%, at least 175%, at least 200%, at most 200%, at most 175%, at most 150%, at most 125%, at most 100%, at most 75%, at most 50%, and/or at most 25% greater than width 1639 in the axial direction of the elliptical band 1620. For example, one or both of apertures 1636 and 1638 may have a length 1637 in the radial direction of the emitter band 1620 that is at least 0.001 inches, at least 0.002 inches, at least 0.003 inches, at least 0.004 inches, at least 0.005 inches, at least 0.006 inches, at least 0.007 inches, at least 0.008 inches, at most 0.020 inches, at most 0.016 inches, at most 0.014 inches, at most 0.012 inches, at most 0.010 inches, at most 0.008 inches, at most 0.007 inches, at most 0.006 inches, at most 0.005 inches, at most 0.004 inches, at most 0.005 inches, at most 0.004 inches, at most 0.003 inches, at most 0.002 inches, and/or at most 0.001 inches. For example, one or both of apertures 1636 and 1638 may have a width 1639 in the axial direction of the emitter band 1620 that is at least 0.001 inches, at least 0.002 inches, at least 0.003 inches, at least 0.004 inches, at least 0.005 inches, at least 0.006 inches, at least 0.007 inches, at least 0.008 inches, at most 0.008 inches, at most 0.007 inches, at most 0.006 inches, at most 0.005 inches, at most 0.004 inches, at most 0.005 inches, at most 0.004 inches, at most 0.003 inches, at most 0.002 inches, and/or at most 0.001 inches.

In some embodiments, an emitter including an elliptical aperture may provide higher sonic output than a comparable emitter including a circular aperture. In some embodiments, an elliptical aperture may help to ensure that the emitter band 1620 has enough material as emitter band material erodes preferentially in the axial direction during shock wave generation. In some embodiments, the continuous ring form factor of the emitter band may enable a more secure attachment of the band to the catheter body.

Emitter band 1620 may include one or more slots in the band for attaching (e.g., soldering) one or more wires to the emitter band. In some examples, enlarged regions 1632 and/or 1634 include one or more slots such as slots or gaps extending into the lateral edges of the respective enlarged region for attaching one or more wires to the emitter band. The one or more slots may be configured such that a wire can be electrically connected (e.g., soldered directly) to the emitter band at one or more of the enlarged regions proximate to the slot(s).

FIG. 16D illustrates a cross-sectional view of a catheter 1600 incorporating a shock wave electrode assembly including emitter band 1620 surrounded by an enclosure 1670 filled with a fluid, where the cross-section is taken across the emitter band 1620. In some embodiments, the emitter band may be embedded or partially embedded within an elongated tube. Embedding the emitter band 1620 at least partially within an elongated tube may provide increased flexibility, for example, because the emitter band 1620 can be smaller in diameter relative to an emitter band that extended wholly outward of the elongated tube. For example, the elongated tube may include an inner layer 1652 and/or an outer layer 1650. For example, outer layer 1650 may be an outer polymer layer and inner layer 1652 may be an inner member. In some embodiments, emitter band 1620 may be encased between outer layer 1650 and inner layer 1652. In some embodiments, at axial sections of the catheter without the emitter band, outer layer 1650 and inner layer 1652 may be indistinguishable from each other. In some embodiments, at axial sections of the catheter without the emitter band, outer layer 1650 and inner layer 1652 may be fused or adhered to each other. In some embodiments, emitter band 1620 may encircle inner layer 1652. In some embodiments, outer layer 1650 may at least partially surround emitter band 1620.

Emitter band 1620 may be separated from one or more electrical wires 1654 by an insulating layer 1656. FIG. 16D depicts insulating layer 1656 as surrounding each of the electrical wires 1654, but in some embodiments, the insulating layer may be formed as an additional layer around inner layer 1652. That is, inner layer 1652 may encapsulate at least a portion of at least one electrical wire 1654. As discussed above, apertures 1636 and/or 1638, and optionally the inner edge of said apertures, can form one electrode in an electrode pair, and a distal end of one or more wires can be positioned near the inner edge of the aperture (e.g. spaced from the inner edge of the aperture by a spark gap) to form the other electrode of an electrode pair. In some examples, the two electrodes may be connected to a pulsed voltage source, such as the voltage source 30 as shown in FIG. 1, such that the electrode pair emits a shock wave when a voltage pulse is applied across the electrode pair.

In some examples, when the catheter has been assembled, the outer layer 1650 overlays at least a portion of the outer surface of the emitter band 1620. For instance, a thin layer of the outer layer 1650 may surround the outer surface of the emitter band 1620 or a portion of the outer surface of the emitter band 1620. The diameter of the emitter band 1620 may be less than the diameter of the outer surface of the outer layer 1650.

Inner layer 1652 and/or outer layer 1650 may be fabricated as an extruded polymer tube. In some examples, inner layer 1652 and/or outer layer 1650 may be formed from a compliant polymeric material, such as such as a polytetrafluoroethylene (PTFE) (e.g., Teflon), PEBA (e.g., Pebax), nylon, urethane, or some other polymeric material. In some examples, the inner layer 1652 and outer layer 1650 are formed from the same polymeric material. However, in other examples, inner layer 1652 is formed of a first polymer or polymeric material, outer layer 1650 is formed of a second polymer or polymeric material different from the first polymeric material. For instance, to facilitate heat sealing of outer layer 1650 over the inner layer 1652, in some examples the first polymer or polymeric material has a higher melting temperature than the second polymer or polymeric material. In such examples, the heat-sealing of outer layer 1650 over inner layer 1652 does not deform or change the diameter of inner layer 1652. In a particular example, the inner layer 1652 is formed from a fluoropolymer (e.g., PTFE), while the outer layer 1650 is formed from a PEBA material.

Inner layer 1652 of the elongated tube may include a guide wire lumen or central lumen 1658 that may accommodate a guide wire, allowing the guide wire to be inserted through the catheter. This in turn may allow a clinician to steer and/or position the catheter at the treatment site. Further, with a guide wire located within central lumen 1658 of catheter 1600, the catheter may be stabilized while one or more shock waves are generated during treatment of calcified lesions within a body lumen.

As discussed in the context of FIGS. 6 and 8, one or more emitter bands 1620 may form first electrodes of a plurality of electrode pairs. In some embodiments, one or more wires positioned proximate to the one or more emitter bands 1620 may form second electrodes of the plurality of electrode pairs. When a voltage pulse is applied across the electrodes of each electrode pair, a shock wave may be generated at the spark gap between the electrodes.

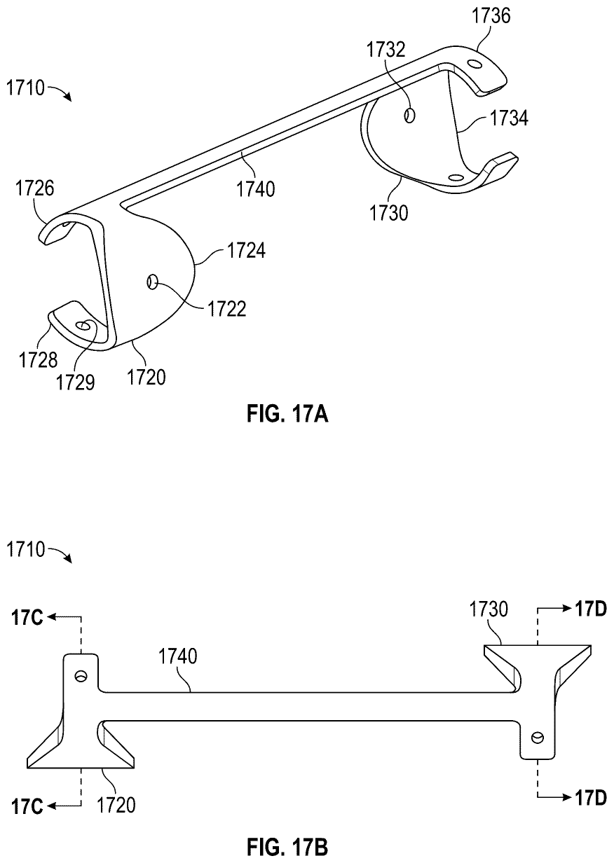

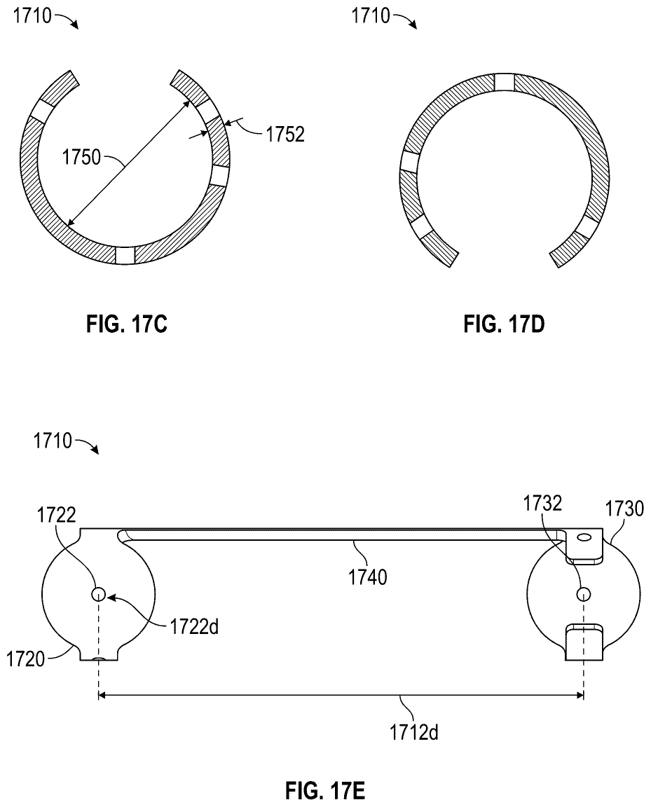

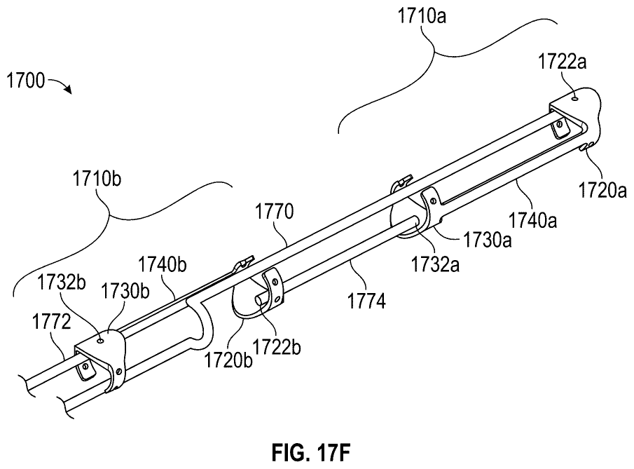

FIGS. 17A-17E illustrate various views of a bridged emitter band 1710. FIG. 17F illustrates a shock wave emitter assembly 1700 including a pair of bridged emitter bands that may be part of a shock wave catheter. As depicted in FIG. 17A, bridged emitter band 1710 may include a pair of discontinuous emitter bands, for example a distal band 1720 and a proximal band 1730, connected by a connecting region or bridge 1740. Each of the pair of discontinuous emitter bands may include an enlarged central region. In some examples, the width or maximum width of the enlarged central region of at least one of the pair of discontinuous emitter bands is greater than the width or maximum width of the connecting region. Distal band 1720 may include an aperture 1722 located on an enlarged region 1724. Proximal band 1730 may include an aperture 1732 extending through enlarged region 1734. Distal band 1720 and/or proximal band 1730 may include one or more leg regions, for example leg regions 1726 and/or 1728 of distal band 1720. Leg regions of distal band 1720 and/or proximal band 1730, for example leg regions 1726 and/or 1728 of distal band 1720, may each include one or more securement structures 1729. Securement structures 1729 may, similar to the securement structures described in the context of FIGS. 13A and 13B, provide structural support to help secure the emitter band to the catheter body. Connecting region or bridge 1740 may be fixedly joined, for example by forming bridged emitter band 1710 out of a single component, to one or more of the leg regions of distal band 1720 and/or proximal band 1730. For example, bridge 1740 may be fixedly joined to leg region 1726 of distal band 1720 and leg region 1736 of proximal band 1730.

As shown in FIG. 17F, distal band 1720a and proximal band 1730a may include apertures 1722a and 1732a, respectively. Aperture 1722a and/or aperture 1732a may form an elliptical shape (any apertures of bands described herein may be elliptically shaped). Aperture 1722a and/or aperture 1732a may be located proximate to an uninsulated region of electrical wires (e.g. first wire 1770, second wire 1772, and/or third wire 1774). An uninsulated region of an electrical wire may form a first electrode of an electrode pair and a conductive surface of the aperture may form a second electrode of the electrode pair, where the electrode pair includes a spark gap where a shock wave may be generated when a voltage pulse is applied across the spark gap. FIG. 17B depicts a side view of bridged emitter band 1710, including distal band 1720 and proximal band 1730 connected by bridge 1740.

FIGS. 17C and 17D depict cross-sectional views of bridged emitter band 1710. As shown in FIG. 17C, bridged emitter band 1710 may have an inner diameter 1750 that is at least 0.01 inches, at least 0.015 inches, at least 0.02 inches, at least 0.025 inches, at least 0.03 inches, at least 0.035 inches, at least 0.04 inches, at most 0.04 inches, at most 0.035 inches, at most 0.03 inches, at most 0.025 inches, at most 0.02 inches, at most 0.015 inches, and/or at most 0.01 inches. Bridged emitter band 1710 may have a thickness 1752 that is at least 0.0005 inches, at least 0.001 inches, at least 0.0015 inches, at least 0.002 inches, at least 0.0025 inches, at least 0.003 inches, at least 0.0035 inches, at least 0.004 inches, at most 0.004 inches, at most 0.0035 inches, at most 0.003 inches, at most 0.0025 inches, at most 0.002 inches, at most 0.0015 inches, at most 0.001 inches, and/or at most 0.0005 inches.

FIG. 17E depicts a side view of bridged emitter band 1710, including distal band 1720 and proximal band 1730 connected by bridge 1740. Distal band 1720 and/or proximal band 1730 may include one or more apertures, for example aperture 1722 and/or aperture 1732. For example, one or both of apertures 1722 and 1732 may have a diameter, for example diameter 1722d, that is at least 0.001 inches, at least 0.002 inches, at least 0.003 inches, at least 0.004 inches, at least 0.005 inches, at least 0.006 inches, at least 0.007 inches, at least 0.008 inches, at most 0.008 inches, at most 0.007 inches, at most 0.006 inches, at most 0.005 inches, at most 0.004 inches, at most 0.005 inches, at most 0.004 inches, at most 0.003 inches, at most 0.002 inches, and/or at most 0.001 inches. Bridged emitter band 1710 may have a length, for example length 1712d, that is at least 0.05, at least 0.075, at least 0.1, at least 0.11, at least 0.125, at least 0.15, at least 0.175, at least 0.2, at least 0.25, at most 0.25, at most 0.2, at most 0.175, at most 0.15, at most 0.125, at most 0.11, at most 0.1, at most 0.075, and/or at most 0.05.

Electrode assembly 1700 depicted in FIG. 17F may be generally similar to the electrode assembly shown in FIG. 8, except that the headphone-shaped emitter bands are replaced with the bridged emitter bands of FIGS. 17A-17E. As seen in FIG. 17F, the electrode assembly 1700 includes a first emitter band 1710a and a second emitter band 1710b that may be connected by first wire 1770, second wire 1772, and/or third wire 1774. As discussed above, an aperture of each of the emitter bands 1710a and/or 1710b can form one electrode in an electrode pair, and a conductive end of one or more of the wires 1770, 1772, and/or 1774 can be positioned near the inner edge of the aperture (e.g. spaced from the inner edge of the aperture by a spark gap) to form the other electrode of an electrode pair. In some examples, the proximal ends of one or more of the wires 1770, 1772, and/or 1774 (e.g. first wire 1770 and the second wire 1772) may be connected to a pulsed voltage source, such as the voltage source 30 as shown in FIG. 1. In a particular example, first wire 1770 is connected to a negative terminal of the voltage source, and the second wire 1772 is connected to a positive terminal or the voltage source or to ground.

As described in relation to FIG. 8, in some examples the ends of one or more of the wires 1770, 1772, and/or 1774 may be positioned apart from the inner edge of the apertures of emitter bands 1710a and/or 1710b by a spark gap, and shock waves may be generated when current flows through the electrode assembly and across one or more spark gaps.

In some implementations, when a voltage is applied across first wire 1770 and second wire 1772, current is configured to flow through the electrode assembly to generate shock waves at the spark gaps between emitter bands 1710a and/or 1710b and conductive ends of the wires 1770, 1772, and/or 1774. For example, in the exemplary electrode assembly 1700, when a voltage pulse is applied across first wire 1770 and second wire 1772, a current may flow from the voltage source through first wire 1770, then from the distal end of first wire 1770 emitter band 1710a and specifically to the inner edge of distal band 1720a. The current may then flow from distal band 1720a to bridge 1740a, and from bridge 1740a to proximal band 1730a. The current may then flow from the inner edge of proximal band 1730a to the distal end of third wire 1774, through third wire 1774, and from the proximal end of third wire 1774 to distal band 1720b of emitter band 1710b. The current may then continue to flow through the rest of the electrode assembly, for example, from the distal band 1720b to bridge 1740b to proximal band 1730b. The current may then flow through the second wire 1772 to reach ground or the positive terminal of a voltage source.

While the bridged emitter band is electrically similar to embodiments of the emitter assembly of FIG. 8 where a pair of emitter bands are in contact with an electrical wire, emitter band 1710 may be included in a catheter when the higher strength of an integrally formed bridged emitter band is favored over emitter bands that are physically connected to each other by, e.g., soldered joints. Additionally, the integrally formed bridged emitter band may provide a slimmer profile than one having soldered joints, which may be bulbous where the joints are formed. Further, an integrally formed bridged emitter band may make manufacturing and/or assembling the catheter simpler and/or easier. By eliminating solder joints, the bridged emitter band may be formed with a lower inner diameter, a lower outer diameter, and/or a lower length, thereby allowing bridged emitter band to more readily be embedded into the catheter body which may enable the manufacture of a smooth-surfaced, low-profile, flexible catheter assembly. Addition of bridge 1740 may additionally help anchor the pair of discontinuous emitter bands to the catheter during use.

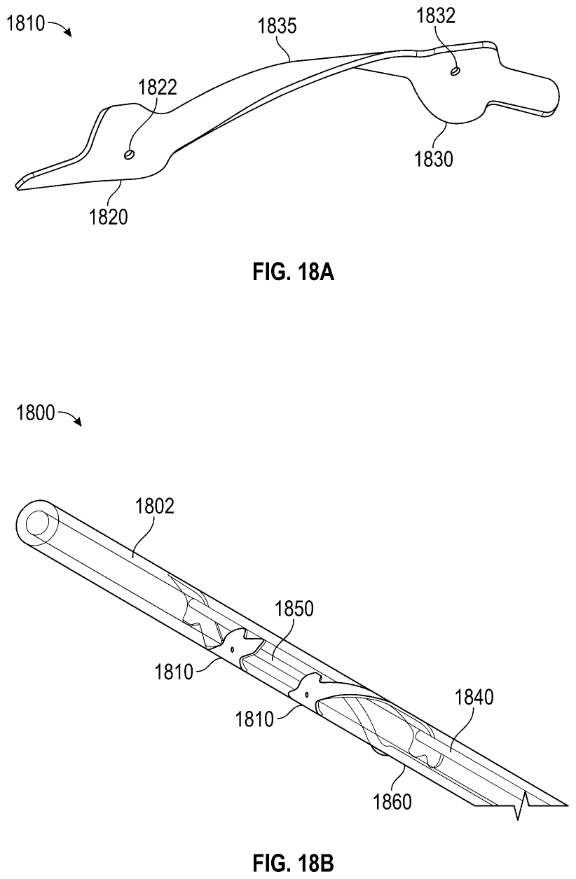

FIG. 18A illustrates an emitter band 1810 that may be included as part of an emitter assembly 1800, illustrated in FIG. 18B. Emitter band 1810 may be similar to the discontinuous spiral emitter band shown in FIG. 5B in that the emitter band may form a coil shape, for example a helical or spiral coil shape, designed to be coiled around (or encapsulated and/or embedded in) an inner shaft 1802 of the catheter. For example, emitter band 1810 may partially encircle an inner layer of the elongated tube. Emitter band 1810 may include enlarged regions 1820 and/or 1830, that may be connected therebetween by a connecting region 1825. In some examples, the width or maximum width of at least one of the enlarged regions 1820 and/or 1830, is greater than the width or maximum width of connecting region 1825. Enlarged regions 1820 and/or 1830 may include apertures 1822 and/or 1832, respectively. Apertures 1822 and/or 1832 may form an elliptical shape. As shown in FIG. 18B, apertures 1822 and/or 1832 may each be located proximate uninsulated regions at the ends of electrical wires 1840, 1850, and/or 1860. Enlarged regions 1820 and/or 1830 may help to ensure structural integrity of the emitter band 1810 after repeated pulsing and potential erosion of apertures 1822 and/or 1832 during shock wave generation. Compared to the bridged emitter band 1710, the emitter band 1810 may be more flexible, leading to more flexibility of a catheter incorporating emitter band 1810.

### EXEMPLARY EMBODIMENTS

Exemplary embodiments of the shock wave catheter systems and methods described herein include:

Embodiment 1. A catheter for treating an occlusion in a body lumen, the catheter comprising:
an elongated tube;
a shock wave electrode assembly at least partially embedded within the elongated tube, the electrode assembly comprising:
   at least one discontinuous emitter band, the at least one emitter band forming a first electrode of an electrode pair, and
   at least one wire, a distal end of the at least one wire forming a second electrode of the electrode pair,
   wherein the shock wave electrode assembly is configured to emit a shock wave when a voltage pulse is applied across the electrode pair; and
an enclosure surrounding the shock wave electrode assembly.

Embodiment 2. The catheter of embodiment 1, wherein discontinuity of the at least one emitter band enables the at least one emitter band to have circumferential flexibility.

Embodiment 3. The catheter of embodiment 1, wherein the emitter band comprises a first end and a second end separated by a gap, and wherein a connecting region between the first end and the second end at least partially encircles the inner layer.

Embodiment 4. The catheter of embodiment 1, wherein the emitter band encircles at least 180 degrees of the inner layer.

Embodiment 5. The catheter of embodiment 1, wherein the emitter band comprises an outer edge, and wherein the outer edge of the emitter band forms the first electrode of the electrode pair.

Embodiment 6. The catheter of embodiment 5, wherein the distal end of the wire is positioned proximate to the outer edge of the emitter band.

Embodiment 7. The catheter of embodiment 1, wherein the emitter band comprises a first enlarged region, a second enlarged region, and a connecting region extending therebetween.

Embodiment 8. The catheter of embodiment 7, wherein the emitter band comprises an aperture extending through the first enlarged region.

Embodiment 9. The catheter of embodiment 8, wherein an inner edge of the aperture forms the first electrode of the electrode pair.

Embodiment 10. The catheter of embodiment 9, wherein the distal end of the wire is positioned proximate to the inner edge of the aperture.

Embodiment 11. The catheter of embodiment 1, wherein the elongated tube comprises an inner layer and an outer layer, the outer layer at least partially surrounding the emitter band.

Embodiment 12. The catheter of embodiment 11, wherein a hole in the outer layer provides a spark gap between the first electrode and the second electrode of the electrode pair.

Embodiment 13. The catheter of embodiment 11, wherein the outer layer overlays at least a portion of an outer surface of the emitter band.

Embodiment 14. The catheter of embodiment 11, wherein the outer layer encapsulates at least a portion of the wire.

Embodiment 15. The catheter of embodiment 11, wherein an outer diameter of the outer layer is equal to an outer diameter of the emitter band.

Embodiment 16. The catheter of embodiment 11, wherein the inner layer is formed of a first polymer, the outer layer is formed of a second polymer, and the first polymer has a higher melting temperature than the second polymer.

Embodiment 17. The catheter of embodiment 1, wherein the emitter band forms electrodes of a plurality of electrode pairs.

Embodiment 18. The catheter of embodiment 1, wherein the elongated tube comprises a central lumen for accommodating a guide wire.

Embodiment 19. A system for treating an occlusion in a body lumen, the system comprising:
the catheter of any of embodiments 1-18; and
a voltage pulse generator electrically connected to the shock wave electrode assembly and configured for applying voltage pulses to the electrode pair to generate shock waves.

Embodiment 20. A method of treating an occlusion in a body lumen, the method comprising:
introducing the catheter of any of embodiments 1-18 into the body lumen;
advancing the catheter through the body lumen to a position proximate to the occlusion;
applying voltage pulses across the electrode pair to generate shock waves inside the enclosure to treat the occlusion.

Embodiment 21. A method of fabricating a catheter, the method comprising:
mounting a shock wave electrode assembly over a first tube, the shock wave electrode assembly comprising:
   at least one wire, a distal end of the at least one wire forming a second electrode of the electrode pair;
   sliding a second tube over the first tube and the shock wave electrode assembly such that the second tube at least partially surrounds the first tube and the shock wave electrode assembly; and
applying an external stimulus to at least partially embed at least a portion of the shock wave electrode assembly within one or both of the first tube and the second tube.

Embodiment 22. The method of embodiment 21, wherein the first tube is an extruded or dip coated polymer tube.

Embodiment 23. The method of embodiment 21, wherein the second tube is an extruded or dip coated polymer tube.

Embodiment 24. The method of embodiment 21, wherein the first tube is formed of a first polymer, the second tube is formed of a second polymer, and the first polymer has a higher melting temperature than the second polymer.

Embodiment 25. The method of embodiment 21, further comprising removing a portion of the second tube to create a spark gap between the first electrode and the second electrode.

Embodiment 26. The method of embodiment 21, further comprising removing a portion of the second tube such that an outer surface of at least a portion of the second tube aligns with an outer surface of the emitter band.

Embodiment 27. The method of embodiment 21, further comprising mounting an enclosure over the second tube such that the enclosure surrounds the shock wave electrode assembly.

Embodiment 28. The method of embodiment 21, wherein the external stimulus comprises heat.

Embodiment 29. The method of embodiment 21, wherein applying the external stimulus causes the second tube to adhere to the first tube such that at least a portion of the shock wave electrode assembly is embedded between the first tube and the second tube.

Embodiment 30. A catheter system for treating an occlusion in a body lumen comprising:
a catheter comprising, at a distal end portion of the catheter:
an inner catheter body comprising an elongate tube.

Embodiment 31. The catheter system of embodiment 30, wherein the leg region comprises a pair of leg regions, each leg region of the pair of leg regions extending from the enlarged region.

Embodiment 32. The catheter system of embodiment 31, wherein each leg region of the pair of leg regions has a leg end and the leg ends do not connect to each other.

Embodiment 33. The catheter system of embodiment 31, wherein each leg region of the pair of leg regions has a leg end and the leg ends overlap each other.

Embodiment 34. The catheter system of embodiment 31, wherein each leg region of the pair of leg regions is substantially a same length.

Embodiment 34. The catheter system of embodiment 31, wherein a first leg region of the pair of leg regions has a first length, a second leg region of the pair of leg regions has a second length, and the first length is different from the second length.

The systems, catheters, and methods described herein have been discussed primarily in the context of treating coronary indications, such as lesions in vasculature, the catheter devices described herein can be used for a variety of indications. For instance, similar designs and methods could be used for treating soft tissues, such as cancer and tumors (i.e., non-thermal ablation methods), blood clots, fibroids, cysts, organs, scar and fibrotic tissue removal, or other tissue destruction and removal treatments. Catheter devices and methods could be used for neurostimulation treatments, targeted drug delivery, treatments of tumors in body lumens (e.g., tumors in blood vessels, the esophagus, intestines, stomach, or vagina), wound treatment, non-surgical removal, and destruction of tissue, or used in place of thermal treatments or cauterization for venous insufficiency and fallopian ligation (i.e., for permanent female contraception). Further, the catheter devices and methods described herein could also be used for tissue engineering methods, for instance, for mechanical tissue decellularization to create a bioactive scaffold in which new cells (e.g., exogenous and endogenous cells) can replace the old cells; introducing porosity to a site to improve cellular retention, cellular infiltration/migration, and diffusion of nutrients and signaling molecules to promote angiogenesis, cellular proliferation, and tissue regeneration similar to cell replacement therapy. Such tissue engineering methods may be useful for treating ischemic heart disease, fibrotic liver, fibrotic bowel, and traumatic spinal cord injury (SCI). For instance, for the treatment of spinal cord injury, the devices and assemblies described herein could facilitate the removal of scarred spinal cord tissue, which acts like a barrier for neuronal reconnection, before the injection of an anti-inflammatory hydrogel loaded with lentivirus to genetically engineer the spinal cord neurons to regenerate.

It should be noted that the elements and features of the example catheters illustrated throughout this specification and drawings may be rearranged, recombined, and modified without departing from the present disclosure. For instance, while this specification and drawings describe and illustrate catheters having certain exemplary emitter band designs, the present disclosure is intended to include catheters having a variety of emitter band configurations. The number, placement, and spacing of the electrode pairs of the electrode assembly can be modified and the number, placement, and spacing of the emitter bands can be modified without departing from the present disclosure.

It will be understood that the foregoing is only illustrative, and that various modifications, alterations and combinations can be made by those skilled in the art without departing from the scope and spirit of the disclosure. Any of the variations of the various catheters disclosed herein can include features described by any other catheters or combination of catheters herein. Furthermore, any of the methods can be used with any of the catheters disclosed. Accordingly, it is not intended that the systems, catheters, and methods described herein be limited, except as by the appended claims.

1. A catheter for treating an occlusion in a body lumen, the catheter comprising:
an elongated tube;
a shock wave electrode assembly at least partially embedded within the elongated tube, the electrode assembly comprising:
at least one discontinuous emitter band, the at least one emitter band forming a first electrode of an electrode pair, and
at least one wire, a distal end of the at least one wire forming a second electrode of the electrode pair,
wherein the shock wave electrode assembly is configured to emit a shock wave when a voltage pulse is applied across the electrode pair; and
an enclosure surrounding the shock wave electrode assembly.

2. The catheter of claim 1, wherein discontinuity of the at least one discontinuous emitter band enables the at least one emitter band to have circumferential flexibility.

3. The catheter of claim 1, wherein the at least one discontinuous emitter band comprises a first end and a second end separated by a gap, and wherein a connecting region between the first end and the second end at least partially encircles an inner layer of the elongated tube.

4. The catheter of claim 3, wherein the at least one discontinuous emitter band encircles at least 180 degrees of the inner layer.

5. The catheter of claim 1, wherein the at least one discontinuous emitter band comprises an outer edge, and wherein the outer edge of the at least one discontinuous emitter band forms the first electrode of the electrode pair.

6. The catheter of claim 5, wherein the distal end of the at least one wire is positioned proximate to the outer edge of the at least one discontinuous emitter band.

7. The catheter of claim 1, wherein the at least one discontinuous emitter band comprises a first enlarged region, a second enlarged region, and at least one connecting region extending therebetween.

8. The catheter of claim 7, wherein a width of at least one of the first enlarged region and the second enlarged region is greater than a width of the at least one connecting region.

9. The catheter of claim 7, wherein the at least one discontinuous emitter band comprises an aperture extending through the first enlarged region.

10. The catheter of claim 9, wherein the aperture has an elliptical shape.

11. The catheter of claim 9, wherein an edge of the aperture forms the first electrode of the electrode pair.

12. The catheter of claim 11, wherein the distal end of the at least one wire is positioned proximate to the edge of the aperture.

13. The catheter of claim 7, wherein the at least one discontinuous emitter band forms a spiral coil shape.

14. The catheter of claim 7, wherein at least one of the first enlarged region or the second enlarged region comprises one or more slots for attaching one or more wires to the at least one discontinuous emitter band.

15. The catheter of claim 1, wherein the elongated tube comprises an inner layer and an outer layer, the outer layer at least partially surrounding the at least one discontinuous emitter band.

16. The catheter of claim 15, wherein a hole in the outer layer provides a spark gap between the first electrode and the second electrode of the electrode pair.

17. The catheter of claim 15, wherein the outer layer overlays at least a portion of an outer surface of the at least one discontinuous emitter band.

18. The catheter of claim 15, wherein the outer layer encapsulates at least a portion of the at least one wire.

19. The catheter of claim 15, wherein an outer diameter of the outer layer is equal to an outer diameter of the at least one discontinuous emitter band.

20. The catheter of claim 15, wherein the inner layer is formed of a first polymer, the outer layer is formed of a second polymer, and the first polymer has a higher melting temperature than the second polymer.

21. The catheter of claim 1, wherein the at least one discontinuous emitter band forms electrodes of a plurality of electrode pairs.

22. The catheter of claim 1, wherein the elongated tube comprises a central lumen for accommodating a guide wire.

23. A system for treating an occlusion in a body lumen, the system comprising:
the catheter of claim 1; and
a voltage pulse generator electrically connected to the shock wave electrode assembly and configured for applying voltage pulses to the electrode pair to generate shock waves.

24. A method of treating an occlusion in a body lumen, the method comprising:
introducing a catheter into the body lumen, the catheter comprising:
an elongated tube,
a shock wave electrode assembly at least partially embedded within the elongated tube, the electrode assembly comprising:
at least one discontinuous emitter band, the at least one emitter band forming a first electrode of an electrode pair, and
at least one wire, a distal end of the at least one wire forming a second electrode of the electrode pair,
wherein the shock wave electrode assembly is configured to emit a shock wave when a voltage pulse is applied across the electrode pair, and
an enclosure surrounding the shock wave electrode assembly;
advancing the catheter through the body lumen to a position proximate to the occlusion;
applying voltage pulses across the electrode pair to generate shock waves inside the enclosure to treat the occlusion.

25. A method of fabricating a catheter, the method comprising:
mounting a shock wave electrode assembly over a first tube, the shock wave electrode assembly comprising:
at least one emitter band, the at least one emitter band forming a first electrode of an electrode pair, and
at least one wire, a distal end of the at least one wire forming a second electrode of the electrode pair;
sliding a second tube over the first tube and the shock wave electrode assembly such that the second tube at least partially surrounds the first tube and the shock wave electrode assembly; and
applying heat to cause the second tube to adhere to the first tube such that at least a portion of the shock wave electrode assembly is at least partially embedded within the second tube.

26. A catheter system for treating an occlusion in a body lumen comprising:
a catheter comprising, at a distal end portion of the catheter:
an inner catheter body,
at least one shock wave electrode pair comprising at least one discontinuous emitter band having an enlarged central region and one or more leg regions extending from the enlarged central region, the one or more leg regions at least partially encircling a circumference of the inner catheter body, and a wire located adjacent the at least one discontinuous emitter band,
an outer catheter body at least partially encapsulating the at least one discontinuous emitter band, and
an enclosure in fluid communication with a fluid lumen of the catheter and surrounding the at least one shock wave electrode pair; and
a power source electrically connected to the wire and configured to apply a voltage across the electrode pair to generate a shock wave within the enclosure.

27. A catheter for treating an occlusion in a body lumen, the catheter comprising:
an elongated tube comprising an inner layer and an outer layer;
a shock wave electrode assembly at least partially embedded within the elongated tube, the electrode assembly comprising:
at least one emitter band comprising an enlarged region and an aperture extending through the enlarged region, an edge of the aperture of the at least one emitter band forming a first electrode of an electrode pair, and
at least one wire, a distal end of the at least one wire forming a second electrode of the electrode pair such that the electrode pair emits a shock wave when a voltage pulse is applied across the electrode pair,
wherein the at least one emitter band encircles the inner layer and the outer layer at least partially surrounds the at least one emitter band; and
an enclosure surrounding the shock wave electrode assembly.

28. A catheter for treating an occlusion in a body lumen, the catheter comprising:
an elongated tube comprising an inner layer and an outer layer;
a shock wave electrode assembly at least partially embedded within the elongated tube, the electrode assembly comprising:
an emitter band that forms a first electrode of an electrode pair, and
a wire including a distal end that forms a second electrode of the electrode pair,
wherein the emitter band at least partially encircles the inner layer and the outer layer at least partially surrounds the emitter band,
wherein the shock wave electrode assembly is configured to emit a shock wave when a voltage pulse is applied across the electrode pair; and
an enclosure surrounding the shock wave electrode assembly.
